(19) United States
(12) Patent Application Publication
(10) Pub. No.: US 2026/0256596 A1
(43) Pub. Date: Sep. 3, 2026

(54) PATIENT-SPECIFIC CERVICAL IMPLANTS AND METHODS OF MAKING THE SAME

(71) Applicant: Carlsmed, Inc., Carlsbad, CA (US)

(72) Inventors: Pedro Resendiz CHAVEZ, Carlsbad, CA (US); Niall Patrick CASEY, Carlsbad, CA (US)

(21) Appl. No.: 19/395,762

(22) Filed: Nov. 20, 2025

Related U.S. Application Data
(60) Provisional application No. 63/723,022, filed on Nov. 20, 2024.

(51) Int. Cl. A61F 2/44; A61F 2/30; G06F 30/20

(52) U.S. Cl. CPC A61F 2/447; G06F 30/20; A61F 2002/30266; A61F 2002/30579; A61F 2002/30948; A61F 2002/30952

(57) ABSTRACT

The present technology includes patient-specific spinal fusion devices, including interbody implants that are designed to be positioned within a disc space between two vertebral bodies. The interbody implants can include a superior-facing endplate and an inferior-facing endplate each having a patient-specific topography. The superior-facing endplate and the inferior-facing endplate can also have different sizes or footprints based on a size and desired implant coverage of the corresponding vertebral body endplate each implant endplate is designed to contact when implanted. Methods for designing and manufacturing patient-specific spinal fusion devices are also described herein.

### CROSS-REFERENCE TO RELATED APPLICATION

The present application claims priority to U.S. provisional patent application No. 63/723,022, filed Nov. 20, 2024, the disclosure of which is incorporated by reference herein in its entirety.

### TECHNICAL FIELD

The present disclosure is generally related to medical care, and more particularly to patient-specific medical implants, including systems and methods designing and manufacturing the same.

### BACKGROUND

Surgical procedures to implant orthopedic implants are used to correct numerous different maladies in a variety of contexts, including spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma. Spine surgery itself may encompass a variety of procedures and targets, such as one or more of the cervical spine, thoracic spine, lumbar spine, or sacrum, and may be performed to treat a deformity or degeneration of the spine and/or related back pain, leg pain, or other body pain. Common spinal deformities that may be treated using an orthopedic implant include irregular spinal curvature such as scoliosis, lordosis, or kyphosis (hyper- or hypo-), and irregular spinal displacement (e.g., spondylolisthesis). Other spinal disorders that can be treated using an orthopedic implant include osteoarthritis, lumbar degenerative disc disease or cervical degenerative disc disease, lumbar spinal stenosis, and cervical spinal stenosis.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a network connection diagram illustrating a system for providing patient-specific medical care in accordance with embodiments of the present technology.

FIG. 2 illustrates a computing device suitable for use in connection with the system of FIG. 1 in accordance with embodiments of the present technology.

FIG. 3 is a flow diagram illustrating a method for providing patient-specific medical care in accordance with embodiments of the present technology.

FIG. 4 is a front view of a patient-specific spinal fusion device configured in accordance with select embodiments of the present technology.

FIG. 5 is an anterior view of the patient-specific spinal fusion device of FIG. 4 implanted in a patient's spine in accordance with select embodiments of the present technology.

FIGS. 6A-6C are end views of adjacent vertebral endplates of a patient's spine.

FIGS. 7A and 7B are front views of another patient-specific spinal fusion device configured in accordance with select embodiments of the present technology.

FIGS. 8A and 8B are front views of yet another patient-specific spinal fusion device configured in accordance with select embodiments of the present technology.

FIG. 9A is a front view of a patient-specific corpectomy device configured in accordance with select embodiments of the present technology, and FIG. 9B is an anterior view of the patient-specific corpectomy device of FIG. 9A implanted in the patient's spine in accordance with select embodiments of the present technology.

FIG. 10 is an anterior view of another patient-specific spinal fusion device including a plate implanted in a patient's spine and configured in accordance with select embodiments of the present technology.

FIGS. 11A-11H illustrate a patient-specific implant system, including a patient-specific interbody implant and a patient-specific anterior plate, configured in accordance with select embodiments of the present technology.

FIGS. 12A-12C illustrate a patient-specific anterior plate assembly configured in accordance with select embodiments of the present technology.

FIGS. 13A and 13B illustrate a patient-specific interbody implant configured in accordance with select embodiments of the present technology.

FIGS. 14A and 14B illustrate another patient-specific interbody implant configured in accordance with select embodiments of the present technology.

FIGS. 15A and 15B illustrate additional patient-specific anterior plate assemblies configured in accordance with select embodiments of the present technology.

FIG. 16 is a flowchart of a method of designing a patient-specific fusion device in accordance with select embodiments of the present technology.

### DETAILED DESCRIPTION

The present technology includes patient-specific spinal fusion devices, including interbody implants that are designed to be positioned within a disc space between two vertebral bodies. The interbody implants can include a superior-facing endplate configured to contact a corresponding first endplate of a first vertebral body, and an inferior-facing endplate configured to contact a corresponding second endplate of a second vertebral body. Both the first and second implant endplates can have a patient-specific topography designed based on the topography of the first and second vertebral body endplates. Further, the superior-facing endplate and the inferior-facing endplate can have different sizes or footprints based on a size and desired implant coverage of the corresponding first and second vertebral body endplates. That is, the size of the implant endplates can be different to account for differences in the size of the vertebral body endplates they are designed to contact when the implant is implanted in the spinal column.

In some embodiments, the present technology includes systems and methods for designing patient-specific surgical plans. This may include, for example, designing patient-specific spinal fusion devices to be implanted in a patient in accordance with the patient-specific surgical plan. Examples of systems and methods for designing patient-specific surgical plans, including designing patient-specific spinal fusion devices, are described in detail throughout this Detailed Description, including in Sections A and B below.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

Although the disclosure herein primarily describes systems and methods for treatment planning in the context of orthopedic surgery, the technology may be applied equally to medical treatment and devices in other fields (e.g., other types of surgical practice). Additionally, although many embodiments herein describe systems and methods with respect to implanted devices, the technology may be applied equally to other types of medical devices (e.g., non-implanted devices).

The headings are provided for convenience only and should not be used to interpret the scope of the present technology.

### A. Select Embodiments of Systems for Designing Patient-Specific Surgical Plans and Patient-Specific Implants

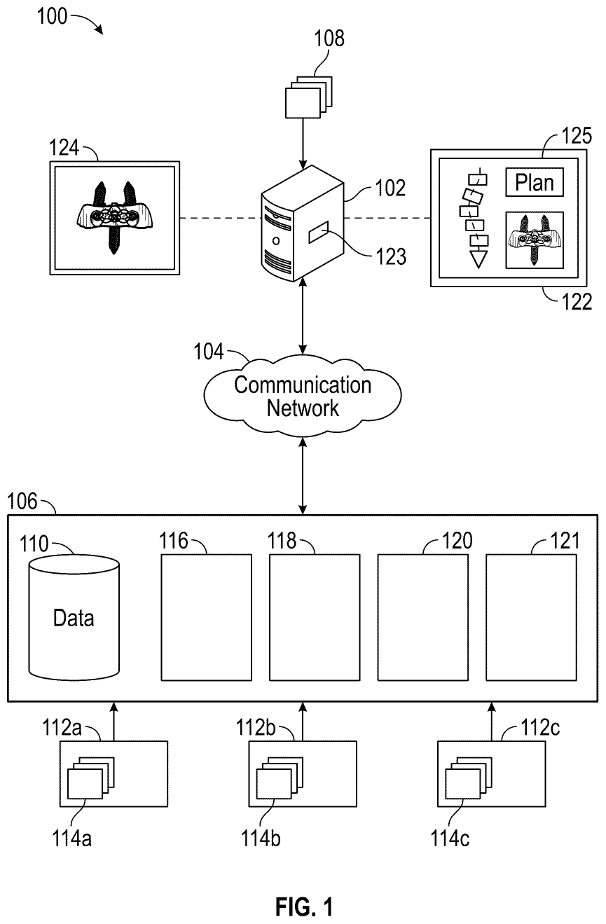

FIG. 1 is a network connection diagram illustrating a system 100 for providing patient-specific medical care, according to embodiments of the present technology. As described in further detail herein, the system 100 is configured to generate a medical treatment plan for a patient. In some embodiments, the system 100 is configured to generate a medical treatment plan for a patient suffering from an orthopedic or spinal disease or disorder, such as trauma (e.g., fractures), cancer, deformity, degeneration, pain (e.g., neck pain, back pain, leg pain), irregular spinal curvature (e.g., scoliosis, lordosis, kyphosis), irregular spinal displacement (e.g., spondylolisthesis, lateral displacement axial displacement), osteoarthritis, cervical degenerative disc disease, lumbar degenerative disc disease, lumbar spinal stenosis, or cervical spinal stenosis, or a combination thereof. The medical treatment plan can include surgical information, technology recommendations (e.g., device and/or instrument recommendations), and/or medical device designs. For example, the medical treatment plan can include at least one surgical procedure (e.g., a surgical procedure or intervention) and/or at least one medical device (e.g., an implanted medical device (also referred to herein as an “implant” or “implanted device”) and/or implant delivery instrument). In some embodiments, the medical treatment plan is therefore also referred to as a “surgical plan,” a “patient-specific surgical plan,” a “patient-specific treatment plan,” or the like.

In some embodiments, the system 100 generates a medical treatment plan that is customized for a particular patient or group of patients, also referred to herein as a “patient-specific” or “personalized” treatment or surgical plan. The patient-specific surgical plan can include at least one patient-specific surgical procedure and/or at least one patient-specific medical device that are designed and/or optimized for the patient's particular characteristics (e.g., condition, anatomy, pathology, medical history, etc.). For example, the patient-specific medical device can be designed and manufactured specifically for the particular patient, rather than being an off-the-shelf device. However, it shall be appreciated that a patient-specific surgical plan can also include aspects that are not customized for the particular patient. For example, a patient-specific or personalized surgical procedure can include one or more instructions, portions, steps, etc. that are non-patient-specific. Likewise, a patient-specific or personalized medical device can include one or more components that are non-patient-specific, and/or can be used with an instrument or tool that is non-patient-specific. Personalized implant designs can be used to manufacture or select patient-specific technologies, including medical devices, instruments, and/or surgical kits. For example, a personalized surgical kit can include one or more patient-specific devices, patient-specific instruments, non-patient-specific technology (e.g., standard instruments, devices, etc.), instructions for use, patient-specific treatment plan information, or a combination thereof.

The system 100 includes a client computing device 102, which can be a user device, such as a smart phone, mobile device, laptop, desktop, personal computer, tablet, phablet, or other such devices known in the art. As discussed further herein, the client computing device 102 can include one or more processors, and memory storing instructions executable by the one or more processors to perform the methods described herein. The client computing device 102 can be associated with a healthcare provider (e.g., a surgeon, healthcare administrator, hospital system, ambulatory surgical centers, etc.) that is treating the patient. Although FIG. 1 illustrates a single client computing device 102, in alternative embodiments, the client computing device 102 can instead be implemented as a client computing system encompassing a plurality of computing devices, such that the operations described herein with respect to the client computing device 102 can instead be performed by the client computing system and/or the plurality of client computing devices.

The client computing device 102 is configured to receive a patient data set 108 associated with a patient to be treated. The patient data set 108 can include data representative of the patient's condition, anatomy, pathology, medical history, preferences, and/or any other information or parameters relevant to the patient. For example, the patient data set 108 can include medical history, surgical intervention data, treatment outcome data, progress data (e.g., physician notes), patient feedback (e.g., feedback acquired using quality of life questionnaires, surveys), clinical data, provider information (e.g., physician, hospital, surgical team), patient information (e.g., demographics, sex, age, height, weight, type of pathology, occupation, activity level, tissue information, health rating, comorbidities, health related quality of life (HRQL)), vital signs, diagnostic results, medication information, allergies, image data (e.g., camera images, Magnetic Resonance Imaging (MRI) images, ultrasound images, Computerized Aided Tomography (CAT) scan images, Positron Emission Tomography (PET) images, X-Ray images), diagnostic equipment information (e.g., manufacturer, model number, specifications, user-selected settings/configurations, etc.), or the like. In some embodiments, the patient data set 108 includes data representing one or more of patient identification number (ID), age, gender, body mass index (BMI), lumbar lordosis, Cobb angle(s), pelvic incidence, disc height, vertebral body height, segment flexibility, bone quality, rotational displacement, and/or treatment level of the spine.

The client computing device 102 is also configured to enable a user (e.g., a surgeon) to review one or more proposed surgical plans for a patient to be treated. In particular, the client computing device 102 can include a surgical plan review software module 123 (“the review module 123”). The review module 123 can comprise computer-executable instructions for generating, displaying, and/or implementing a surgical plan review program or platform 125 (“the review program 125”) that facilitates surgeon or user review of one or more patient-specific surgical plans via the client computing device 102.

The review module 123 can be stored in the form of computer-readable or computer-executable instructions on a memory (not shown) of the client computing device 102. In other embodiments, the review module 123 can be stored remotely from the client computing device 102 (e.g., in the cloud or at a remote server) and implemented on the client computing device 102 via a remote (e.g., wireless) connection. In yet other embodiments, some of the review module 123 can be stored locally at the client computing device 102 while other aspects of the review module 123 can be store remotely. In some embodiments, the review module 123 can be generally similar to the review modules and associated platforms described in U.S. Patent Application Publication No. 2024/0138919, the disclosure of which is incorporated by reference in its entirety.

The client computing device 102 is operably connected via a communication network 104 to a server 106, thus allowing for data transfer between the client computing device 102 and the server 106. The communication network 104 may be a wired and/or a wireless network. The communication network 104, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 106, which may also be referred to as a “treatment assistance network” or “prescriptive analytics network,” can include one or more computing devices and/or systems. As discussed further herein, the server 106 can include one or more processors, and memory storing instructions executable by the one or more processors to perform some or all of the methods described herein. In some embodiments, the server 106 is implemented as a distributed “cloud” computing system or facility across any suitable combination of hardware and/or virtual computing resources.

The client computing device 102 and server 106 can individually or collectively perform some or all of the various methods described herein for providing patient-specific medical care. For example, some or all of the steps of the methods described herein can be performed by the client computing device 102 alone, the server 106 alone, or a combination of the client computing device 102 and the server 106. Thus, although certain operations are described herein with respect to the server 106, it shall be appreciated that these operations can also be performed by the client computing device 102, and vice-versa, unless the context clearly dictates otherwise.

The server 106 includes at least one database 110 configured to store reference data useful for the treatment planning methods described herein. The reference data can include historical and/or clinical data from the same or other patients, data collected from prior surgeries and/or other treatments of patients by the same or other healthcare providers, data relating to medical device designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like.

In some embodiments, the database 110 includes a plurality of reference patient data sets, each reference patient data set associated with a corresponding reference patient. For example, the reference patient can be a patient that previously received treatment or is currently receiving treatment. Each reference patient data set can include data representative of the corresponding reference patient's condition, anatomy, pathology, medical history, disease progression, preferences, and/or any other information or parameters relevant to the reference patient, such as any of the data described herein with respect to the patient data set 108. In some embodiments, the reference patient data set includes pre-operative data, intra-operative data, and/or post-operative data. For example, a reference patient data set can include data representing one or more of patient ID, age, gender, BMI, lumbar lordosis, Cobb angle(s), pelvic incidence, disc height, vertebral body height, segment flexibility, bone quality, rotational displacement, and/or treatment level of the spine. As another example, a reference patient data set can include treatment data regarding at least one surgical procedure performed on the reference patient, such as descriptions of surgical procedures or interventions (e.g., surgical approaches, bony resections, surgical maneuvers, corrective maneuvers, placement of implants or other devices). In some embodiments, the treatment data includes medical device design data for at least one medical device used to treat the reference patient, such as physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties). In yet another example, a reference patient data set can include outcome data representing an outcome of the treatment of the reference patient, such as corrected anatomical metrics, presence of fusion, HRQL, pain level, activity level, return to work, complications, recovery times, efficacy, mortality, and/or follow-up surgeries.

In some embodiments, the server 106 receives at least some of the reference patient data sets from a plurality of healthcare provider computing systems (e.g., systems 112a-112c, collectively 112). The server 106 can be connected to the healthcare provider computing systems 112 via one or more communication networks (not shown). Each healthcare provider computing system 112 can be associated with a corresponding healthcare provider (e.g., physician, surgeon, medical clinic, hospital, healthcare network, etc.). Each healthcare provider computing system 112 can include at least one reference patient data set (e.g., reference patient data sets 114a-114c, collectively 114) associated with reference patients treated by the corresponding healthcare provider. The reference patient data sets 114 can include, for example, electronic medical records, electronic health records, biomedical data sets, etc. The reference patient data sets 114 can be received by the server 106 from the healthcare provider computing systems 112 and can be reformatted into different formats for storage in the database 110. Optionally, the reference patient data sets 114 can be processed (e.g., cleaned) to ensure that the represented patient parameters are likely to be useful in the treatment planning methods described herein.

As described in further detail herein, the server 106 can be configured with one or more algorithms that generate patient-specific surgical plan data (e.g., treatment procedures, target anatomical corrections, medical devices, etc.) based on the reference data. In some embodiments, the patient-specific data is generated based on correlations between the patient data set 108 and the reference data. Optionally, the server 106 can predict outcomes, including recovery times, efficacy based on clinical end points, likelihood of success, predicted mortality, predicted related follow-up surgeries, or the like. In some embodiments, the server 106 can continuously or periodically analyze patient data (including patient data obtained during the patient stay) to determine near real-time or real-time risk scores, mortality prediction, etc.

In some embodiments, the server 106 includes one or more modules for performing one or more steps of the patient-specific treatment planning methods described herein. For example, in the depicted embodiment, the server 106 includes a data analysis module 116, a treatment planning module 118, a disease progression module 120, and an intervention timing module 121. In alternative embodiments, one or more of these modules may be combined with each other, or may be omitted. Thus, although certain operations are described herein with respect to a particular module or modules, this is not intended to be limiting, and such operations can be performed by a different module or modules in alternative embodiments.

The data analysis module 116 is configured with one or more algorithms for identifying a subset of reference data from the database 110 that is likely to be useful in developing a patient-specific treatment plan. For example, the data analysis module 116 can compare patient-specific data (e.g., the patient data set 108 received from the client computing device 102) to the reference data from the database 110 (e.g., the reference patient data sets) to identify similar data (e.g., one or more similar patient data sets in the reference patient data sets). The comparison can be based on one or more parameters, such as age, gender, BMI, lumbar lordosis, pelvic incidence, and/or treatment levels. The parameter(s) can be used to calculate a similarity score for each reference patient. The similarity score can represent a statistical correlation between the patient data set 108 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference is below a threshold can be considered to be similar patients.

The data analysis module 116 can further be configured with one or more algorithms to select a subset of the reference patient data sets, e.g., based on similarity to the patient data set 108 and/or treatment outcome of the corresponding reference patient. For example, the data analysis module 116 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, presence of fusion, HRQL, activity level, complications, recovery times, efficacy, mortality, or follow-up surgeries. As described in further detail below, in some embodiments, the data analysis module 116 calculates an outcome score by assigning values to each outcome parameter. A patient can be considered to have a favorable outcome if the outcome score is above, below, or at a specified threshold value.

In some embodiments, the data analysis module 116 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 116 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular health-care provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and other data sets can be utilized. By way of example, a patient-specific surgical plan to perform a battlefield surgery can be based on reference patient data from similar battlefield surgeries and/or data sets associated with battlefield surgeries. In another example, the patient-specific surgical plan can be generated based on available robotic surgical systems. The reference patient data sets can be selected based on patients that have been operated on using comparable robotic surgical systems under similar conditions (e.g., size and capabilities of surgical teams, hospital resources, etc.).

The treatment planning module 118 is configured with one or more algorithms to generate at least one surgical plan (e.g., pre-operative plans, intra-operative plans, post-operative plans etc.) based on the output from the data analysis module 116. In some embodiments, the treatment planning module 118 is configured to develop and/or implement at least one predictive model for generating the patient-specific treatment plan, also known as a “prescriptive model.” The predictive model(s) can be developed using clinical knowledge, statistics, machine learning, AI, neural networks, or the like. In some embodiments, the output from the data analysis module 116 is analyzed (e.g., using statistics, machine learning, neural networks, AI) to identify correlations between data sets, patient parameters, healthcare provider parameters, healthcare resource parameters, treatment procedures, medical device designs, and/or treatment outcomes. These correlations can be used to develop at least one predictive model that predicts the likelihood that a surgical plan will produce a favorable outcome for the particular patient. The predictive model(s) can be validated, e.g., by inputting data into the model(s) and comparing the output of the model to the expected output.

In some embodiments, the treatment planning module 118 is configured to generate the surgical plan based on previous treatment data from reference patients. For example, the treatment planning module 118 can receive a selected subset of reference patient data sets and/or similar patient data sets from the data analysis module 116, and determine or identify treatment data from the selected subset. The treatment data can include, for example, treatment procedure data (e.g., surgical procedure or intervention data) and/or medical device design data (e.g. implant design data) that are associated with favorable or desired treatment outcomes for the corresponding patient. The treatment planning module 118 can analyze the treatment procedure data and/or medical device design data to determine an optimal treatment protocol for the patient to be treated. For example, the treatment procedures and/or medical device designs can be assigned values and aggregated to produce a treatment score. The patient-specific surgical plan can be determined by selecting surgical plan(s) based on the score (e.g., higher or highest score; lower or lowest score; score that is above, below, or at a specified threshold value). The personalized patient-specific surgical plan can be based on, at least in part, the patient-specific technologies or patient-specific selected technology.

Alternatively or in combination, the treatment planning module 118 can generate the surgical plan based on correlations between data sets. For example, the treatment planning module 118 can correlate treatment procedure data and/or medical device design data from similar patients with favorable outcomes (e.g., as identified by the data analysis module 116). Correlation analysis can include transforming correlation coefficient values to values or scores. The values/scores can be aggregated, filtered, or otherwise analyzed to determine one or more statistical significances. These correlations can be used to determine surgical procedure(s) and/or medical device design(s) that are optimal or likely to produce a favorable outcome for the patient to be treated.

Alternatively or in combination, the treatment planning module 118 can generate the surgical plan using one or more AI techniques. AI techniques can be used to develop computing systems capable of simulating aspects of human intelligence, e.g., learning, reasoning, planning, problem solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the treatment planning module 118 generates the surgical plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 110, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training data set can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate a surgical plan, the patient data set 108 can be input into the trained machine learning model(s). Additional data, such as the selected subset of reference patient data sets and/or similar patient data sets, and/or treatment data from the selected subset, can also be input into the trained machine learning model(s). The trained machine learning model(s) can then calculate whether various candidate treatment procedures and/or medical device designs are likely to produce a favorable outcome for the patient. Based on these calculations, the trained machine learning model(s) can select at least one surgical plan for the patient. In some embodiments, the trained machine learning model(s) can determine candidate procedures (or candidate surgical plans), analyze the candidate procedures, select the candidate surgical plans or portions thereof, score plans, and/or generate surgical plans for the patient. Each surgical plan can be scored (e.g., scored based on favorable outcome, likelihood of outcome, etc.) and ranked according to the score. The trained machine learning model(s) can determine a set of surgical plans that meet selection criteria for plan review by a user. The selection criteria can be based on, for example, regulatory requirements, reimbursement criteria, healthcare/provider expertise, available surgical equipment, manufacturing capabilities, elimination criteria, combinations thereof, or the like. A user can input one or more selection criteria to control the types and/or features of the surgical plans for comparison. In embodiments where multiple trained machine learning models are used, the models can be run sequentially or concurrently to compare outcomes and can be periodically updated using training data sets. The treatment planning module 118 can use one or more of the machine learning models based the model's predicted accuracy score.

The patient-specific surgical plan generated by the treatment planning module 118 can include at least one patient-specific surgical procedure (e.g., a surgical procedure or intervention) and/or at least one patient-specific medical device (e.g., an implant or implant delivery instrument). A patient-specific surgical plan can include an entire surgical procedure or portions thereof. Additionally, one or more patient-specific medical devices can be specifically selected or designed for the corresponding surgical procedure, thus allowing for the various components of the patient-specific technology to be used in combination to treat the patient.

In some embodiments, the patient-specific surgical procedure includes an orthopedic surgery procedure, such as spinal surgery, hip surgery, knee surgery, jaw surgery, hand surgery, shoulder surgery, elbow surgery, total joint reconstruction (arthroplasty), skull reconstruction, foot surgery, or ankle surgery. Spinal surgery can include spinal fusion surgery, such as anterior cervical fusion (ACF), posterior cervical fusion (PCF), posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). Spinal surgery can also include a corpectomy procedure in which a substantial portion of a vertebral body is removed and an implant is inserted that spans multiple vertebral levels. In some embodiments, the patient-specific treatment procedure includes descriptions of and/or instructions for performing one or more aspects of a patient-specific surgical procedure. For example, the patient-specific surgical procedure can include one or more of a surgical approach, a corrective maneuver, a bony resection, or implant placement.

In some embodiments, the patient-specific medical device design includes a design for an orthopedic implant and/or a design for an instrument for delivering an orthopedic implant. Examples of such implants include, but are not limited to, screws (e.g., bone screws, spinal screws, pedicle screws, facet screws), interbody implant devices (e.g., intervertebral implants), cages, plates, rods, disks, fusion devices, spacers, rods, expandable devices, stents, brackets, ties, scaffolds, fixation device, anchors, nuts, bolts, rivets, connectors, tethers, fasteners, joint replacements, hip implants, or the like. Examples of instruments include, but are not limited to, screw guides, cannulas, ports, catheters, insertion tools, removal tools, awls, drivers, or the like.

A patient-specific medical device design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of a corresponding medical device. For example, a design for an orthopedic implant can include implant shape, size, material, and/or effective stiffness (e.g., lattice density, number of struts, location of struts, etc.). In some embodiments, the generated patient-specific medical device design is a design for an entire device. Alternatively, the generated design can be for one or more components of a device, rather than the entire device.

In some embodiments, the design is for one or more patient-specific device components that can be used with standard, off-the-shelf components. For example, in a spinal surgery, a surgical kit can include both standard components and patient-specific customized components. In some embodiments, the surgical kit can include a patient specific interbody device that can be used with standard interfixating screws. In some embodiments, the generated design is for a patient-specific implant that can be used with a standard, off-the-shelf delivery instrument. For example, the implants (e.g., interbody device, screws, screw holders, rods) can be designed and manufactured for the patient, while the instruments for delivering the implants can be standard instruments. This approach allows the components that are implanted to be designed and manufactured based on the patient's anatomy and/or surgeon's preferences to enhance treatment. The patient-specific devices described herein are expected to improve delivery into the patient's body, placement at the treatment site, and/or interaction with the patient's anatomy.

In embodiments in which the patient-specific surgical plan includes a specific surgical procedure to implant a medical device, the treatment planning module 118 can also store various types of implant surgery information, such as implant parameters (e.g., types, dimensions), availability of implants, aspects of a pre-operative plan (e.g., initial implant configuration, detection and measurement of the patient's anatomy, etc.), FDA requirements for implants (e.g., specific implant parameters and/or characteristics for compliance with FDA regulations), or the like. In some embodiments, the treatment planning module 118 can convert the implant surgery information into formats useable for machine-learning based models and algorithms. For example, the implant surgery information can be tagged with particular identifiers for formulas or can be converted into numerical representations suitable for supplying to the trained machine learning model(s). The treatment planning module 118 can also store information regarding the patient's anatomy, such as two- or three-dimensional images or models of the anatomy, and/or information regarding the biology, geometry, and/or mechanical properties of the anatomy. The anatomy information can be used to inform implant design and/or placement.

The disease progression module 120 can be used to analyze, predict, and/or model disease progression for a particular patient. As described in detail below, the disease progression module 120 can estimate the rate of disease progression for the patient under a variety of different circumstances, including (a) if no surgical intervention occurs, and (b) if one or more surgical plans (e.g., surgical procedures identified by the treatment planning module 118) are performed. The disease progression module 120 can therefore include an algorithm, machine learning model, or other software analytical tool for predicting disease progression in a particular patient.

In some embodiments, the disease progression module 120 includes a machine learning model or other software module that can be trained based off a plurality of reference patient data sets that includes, in addition to the patient data described above, disease progression metrics for each of the reference patients. The progression metrics can include measurements for disease metrics over a period of time. Suitable metrics may include spinopelvic parameters (e.g., lumbar lordosis, pelvic tilt, sagittal vertical axis (SVA), cobb angel, coronal offset, etc.), disability scores, functional ability scores, flexibility scores, VAS pain scores, or the like. The progression of the metrics for each reference patient can be correlated to other patient information for the specific reference patient (e.g., age, sex, height, weight, activity level, diet, etc.). The disease metrics can include values over a period of time. For example, the reference patient data may include values of disease metrics on a daily, weekly, monthly, bi-monthly, yearly, or other basis. By measuring the metrics over a period of time, changes in the values of the metrics can be tracked as an estimate of disease progression and correlated to other patient data.

In some embodiments, the disease progression module 120 can therefore estimate the rate of disease progression for a particular patient. The progression may be estimated by providing estimated changes in one or more disease metrics over a period of time (e.g., X% increase in a disease metric per year). The rate can be constant (e.g., 5% increase in pelvic tilt per year) or variable (e.g., 5% increase in pelvic tilt for a first year, 10% increase in pelvic tilt for a second year, etc.). In some embodiments, the estimated rate of progression can be transmitted to a surgeon or other healthcare provider as part of a surgical plan, as described in greater detail below.

As a non-limiting example, a particular patient who is a fifty-five-year-old male may have a SVA value of 6 mm. The disease progression module 120 can analyze patient reference data sets to identify disease progression for individual reference patients having one or more similarities with the particular patient (e.g., individual patients of the reference patients who have an SVA value of about 6 mm and are approximately the same age, weight, height, and/or sex of the patient). Based on this analysis, the disease progression module 120 can predict the rate of disease progression if no surgical intervention occurs (e.g., the patient's VAS pain scores may increase 5%, 10%, or 15% annually if no surgical intervention occurs, the SVA value may continue to increase by 5% annually if no surgical intervention occurs, etc.).

The surgical treatment plans and/or associated patient-specific implants described herein can also be at least partially based on the estimated rates of disease progression, enabling the modeling of different outcomes over a desired period of times. Additionally, the models/simulations can account for any number of additional diseases or conditions to predict the patient's overall health, mobility, or the like. These additional diseases or conditions can, in combination with other patient health factors (e.g., height, weight, age, activity level, etc.) be used to generate a patient health score reflecting the overall health of the patient. The patient health score can be displayed for surgeon review and/or incorporated into the estimation of disease progression. Accordingly, the present technology can generate one or more virtual simulations of the predicted disease progression to demonstrate how the patient's anatomy is predicted to change over time. Physician input can be used to generate or modify the virtual simulation(s). The present technology can generate one or more post-treatment virtual simulations based on the received physician input for review by the healthcare provider, patient, etc.

In some embodiments, the present technology can also predict, model, and/or simulate disease progression based on one or more potential surgical plans. For example, the disease progression module 120 may simulate what a patient's anatomy and/or spinal metrics may be 1, 2, 5, or 10 years post-surgery for several different surgical plans. The simulations may also incorporate non-surgical factors, such as patient age, height, weight, sex, activity level, other health conditions, or the like, as previously described. The system and/or a surgeon can use the disease progression to aid in selecting which surgical plan provides the best long-term efficacy, as described below. These simulations can also be used to determine patient-specific corrections that compensate for the projected diseases progression.

Accordingly, in some embodiments, multiple disease progression models (e.g., two, three, four, five, six, or more) are simulated to provide disease progression data for several different surgical plans. For example, the disease progression module can generate models that predict post-surgical disease progression for each of three different surgical plans. A surgeon or other healthcare provider can review the disease progression models and, based on the review, select which of the three surgical plans is likely to provide the patient with the best long-term outcome.

Based off of the modeled disease progression, the systems and methods described herein can also (i) identify a recommended time for surgical intervention, and/or (ii) identify a recommended type of surgical procedure for the patient. In some embodiments, the present technology therefore includes an intervention timing module 121 that includes an algorithm, machine learning model, or other software analytical tool for determining the optimal time for surgical intervention in a particular patient. This can be done, for example, by analyzing patient reference data that includes (i) pre-operative disease progression metrics for individual reference patients, (ii) disease metrics at the time of surgical intervention for individual reference patients, (iii) post-operative disease progression metrics for individual reference patients, and/or (iv) scored surgical outcomes for individual reference patients. The intervention timing module 121 can compare the disease metrics for a particular patient to the reference patient data sets to determine, for similar patients, the point of disease progression at which surgical intervention produced the most favorable outcomes.

As a non-limiting example, the reference patient data sets may include data associated with reference patients' sagittal vertical axis. The data can include (i) sagittal vertical axis values for individual patients over a period of time before surgical intervention (e.g., how fast and to what degree the sagittal vertical axis value changed), (ii) sagittal vertical axis of the individual patients at the time of surgical intervention, (iii) the change in sagittal vertical axis after surgical intervention, and (iv) the degree to which the surgical intervention was successful (e.g., based on pain, quality of life, or other factors). Based on the foregoing data, the intervention timing module 121 can, based on a particular patient's sagittal vertical axis value, identify at which point surgical intervention will have the highest likelihood of producing the most favorable outcome. Of course, the foregoing metric is provided by way of example only, and the intervention timing module 121 can incorporate other metrics (e.g., lumbar lordosis, pelvic tilt, sagittal vertical axis, cobb angel, coronal offset, disability scores, functional ability scores, flexibility scores, VAS pain scores) instead of or in combination with sagittal vertical axis to predict the time at which surgical intervention has the highest probability of providing a favorable outcome for the particular patient.

The intervention timing module 121 may also incorporate one or more mathematical rules based on value thresholds for various disease metrics. For example, the intervention timing module 121 may indicate surgical intervention is necessary if one or more disease metrics exceed a predetermined threshold or meet some other criteria. Representative thresholds that indicate surgical intervention may be necessary include SVA values greater than 7 mm, a mismatch between lumbar lordosis and pelvic incidence greater than 10 degrees, a cobb angle of greater than 10 degrees, and/or a combination of cobb angle and LL/PI mismatch greater than 20 degrees. Of course, other threshold values and metrics can be used; the foregoing are provided as examples only. In some embodiments, the foregoing rules can be tailored to specific patient populations (e.g., for males over 50 years of age, an SVA value greater than 7 mm indicates the need for surgical intervention). If a particular patient does not exceed the thresholds indicating surgical intervention is recommended, the intervention timing module 121 may provide an estimate for when the patient's metrics will exceed one or more thresholds, thereby providing the patient with an estimate of when surgical intervention may become recommended.

In some embodiments, the treatment planning module 118 identifies one or more types of surgical procedures for the patient based at least in part on the disease progression of the patient determined using the disease progression module 120 and/or the intervention timing module 121. The treatment planning module 118 may also incorporate one or more mathematical rules for identifying surgical procedures. As a non-limiting example, if a LL/PI mismatch is between 10 and 20 degrees, the treatment planning module 118 may recommend an anterior fusion surgery, but if the LL/PI mismatch is greater than 20 degrees, the treatment planning module may recommend both anterior and posterior fusion surgery. As another non-limiting example, if a SVA value is between 7 mm and 15 mm, the treatment planning module may recommend posterior fusion surgery, but if the SVA is above 15 mm, the treatment planning module may recommend both posterior fusion surgery and anterior fusion surgery. Of course, other rules can be used; the foregoing are provided as examples only.

Without being bound by theory, incorporating disease progression modeling into the patient-specific surgical plans described herein may even further increase the effectiveness of the procedures and/or provide a surgeon more data by which to evaluate various surgical plans. For example, in many cases it may be disadvantageous to operate after a patient's disease progresses to an irreversible or unstable state. However, it may also be disadvantageous to operate too early, such as before the patient's disease is causing symptoms and/or if the patient's disease may not progress further. The disease progression module 120 and/or the intervention timing module 121 can therefore help identify the window of time during which surgical intervention in a particular patient has the highest probability of providing a favorable outcome for the patient.

The surgical plan(s) generated by the treatment planning module 118 can be transmitted via the communication network 104 to the client computing device 102 for output to a user (e.g., clinician, surgeon, healthcare provider, patient). In some embodiments, the client computing device 102 includes or is operably coupled to a display 122 for outputting the treatment plan(s). The display 122 can include a graphical user interface (GUI) for visually depicting various aspects of the surgical plan(s). For example, the display 122 can show various aspects of a surgical procedure to be performed on the patient, such as the surgical approach, treatment levels, corrective maneuvers, tissue resection, and/or implant placement. To facilitate visualization, the surgical plan can include a virtual model of the surgical procedure that can be displayed via the display 122. The display 122 may also display additional aspects of the surgical plan, such as predicted post-operative patient metrics, predicted disease progression metrics associated with the identified surgical procedure, etc. As another example, the display 122 can show a design for a medical device to be implanted in the patient in accordance with the transmitted surgical plan, such as a two- or three-dimensional model of the device design. The display 122 can also show patient information, such as two- or three-dimensional images or models of the patient's anatomy where the surgical procedure is to be performed and/or where the device is to be implanted. The client computing device 102 can further include one or more user input devices (not shown) allowing the user to modify, select, approve, and/or reject the displayed treatment plan(s).

In some embodiments, one or more aspects of the surgical plan are displayed using the surgical plan review program 125. For example, the review program 125, which may be implemented as a mobile phone application, a computer application, or the like, can display (e.g., via the display 122) one or more aspects of the surgical plan (e.g., a surgical procedure, a virtual model of patient anatomy, an implant, etc.). The review program 125 may provide an interactive interface that further enables a surgeon to select between different patients, select between different surgical plans for the same patient, compare surgical plans for the same patient, review the status of a surgical plan, provide feedback on a proposed surgical plan, accept a surgical plan, reject a surgical plan, etc. The review program 125 may further enable a surgeon or other user to select between different views of a virtual model of patient anatomy, and/or different views of a patient-specific implant to be used in the surgical plan.

In some embodiments, the medical device design(s) generated by the treatment planning module 118 can be transmitted from the client computing device 102 and/or server 106 to a manufacturing system 124 for manufacturing a corresponding medical device. The manufacturing system 124 can be located on site or off site. On-site manufacturing can reduce the number of sessions with a patient and/or the time to be able to perform the surgery whereas off-site manufacturing can be useful make the complex devices. Off-site manufacturing facilities can have specialized manufacturing equipment. In some embodiments, more complicated device components can be manufactured off site, while simpler device components can be manufactured on site.

Various types of manufacturing systems are suitable for use in accordance with the embodiments herein. For example, the manufacturing system 124 can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system 124 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 124 can manufacture one or more patient-specific medical devices based on fabrication instructions or data (e.g., CAD data, 3D data, digital blueprints, stereolithography data, or other data suitable for the various manufacturing technologies described herein). Different components of the system 100 can generate at least a portion of the manufacturing data used by the manufacturing system 124. The manufacturing data can include, without limitation, fabrication instructions (e.g., programs executable by additive manufacturing equipment, subtractive manufacturing equipment, etc.), 3D data, CAD data (e.g., CAD files), CAM data (e.g., CAM files), path data (e.g., print head paths, tool paths, etc.), material data, tolerance data, surface finish data (e.g., surface roughness data), regulatory data (e.g., FDA requirements, reimbursement data, etc.), or the like. The manufacturing system 124 can analyze the manufacturability of the implant design based on the received manufacturing data. The implant design can be finalized by altering geometries, surfaces, etc. and then generating manufacturing instructions. In some embodiments, the server 106 generates at least a portion of the manufacturing data, which is transmitted to the manufacturing system 124.

The manufacturing system 124 can generate CAM data, print data (e.g., powder bed print data, thermoplastic print data, photo resin data, etc.), or the like and can include additive manufacturing equipment, subtractive manufacturing equipment, thermal processing equipment, or the like. The additive manufacturing equipment can be 3D printers, stereolithography devices, digital light processing devices, fused deposition modeling devices, selective laser sintering devices, selective laser melting devices, electronic beam melting devices, laminated object manufacturing devices, powder bed printers, thermoplastic printers, direct material deposition devices, or inkjet photo resin printers, or like technologies. The subtractive manufacturing equipment can be CNC machines, electrical discharge machines, grinders, laser cutters, water jet machines, manual machines (e.g., milling machines, lathes, etc.), or like technologies. Both additive and subtractive techniques can be used to produce implants with complex geometries, surface finishes, material properties, etc. The generated fabrication instructions can be configured to cause the manufacturing system 124 to manufacture the patient-specific orthopedic implant that matches or is therapeutically the same as the patient-specific design. In some embodiments, the patient-specific medical device can include features, materials, and designs shared across designs to simplify manufacturing. For example, deployable patient-specific medical devices for different patients can have similar internal deployment mechanisms but have different deployed configurations. In some embodiments, the components of the patient-specific medical devices are selected from a set of available pre-fabricated components and the selected pre-fabricated components can be modified based on the fabrication instructions or data.

The surgical plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments the treatment planning module 118 generates control instructions configured to cause a surgical robot (e.g., robotic surgery systems, navigation systems, etc.) to partially or fully perform a surgical procedure. The control instructions can be transmitted to the robotic apparatus by the client computing device 102 and/or the server 106.

Following the treatment of the patient in accordance with the surgical plan, treatment progress can be monitored over one or more time periods to update the data analysis module 116, treatment planning module 118, disease progression module 120, and/or intervention timing module 121. Post-treatment data can be added to the reference data stored in the database 110. The post-treatment data can be used to train machine learning models for developing patient-specific treatment plans, patient-specific medical devices, or combinations thereof.

It shall be appreciated that the components of the system 100 can be configured in many different ways. For example, in alternative embodiments, the database 110, the data analysis module 116, the treatment planning module 118, the disease progression module 120, and/or the intervention timing module 121 can be components of the client computing device 102, rather than the server 106. As another example, the database 110, the data analysis module 116, the treatment planning module 118, the disease progression module 120, and/or the intervention timing module 121 can be located across a plurality of different servers, computing systems, or other types of cloud-computing resources, rather than at a single server 106 or client computing device 102.

Additionally, in some embodiments, the system 100 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like.

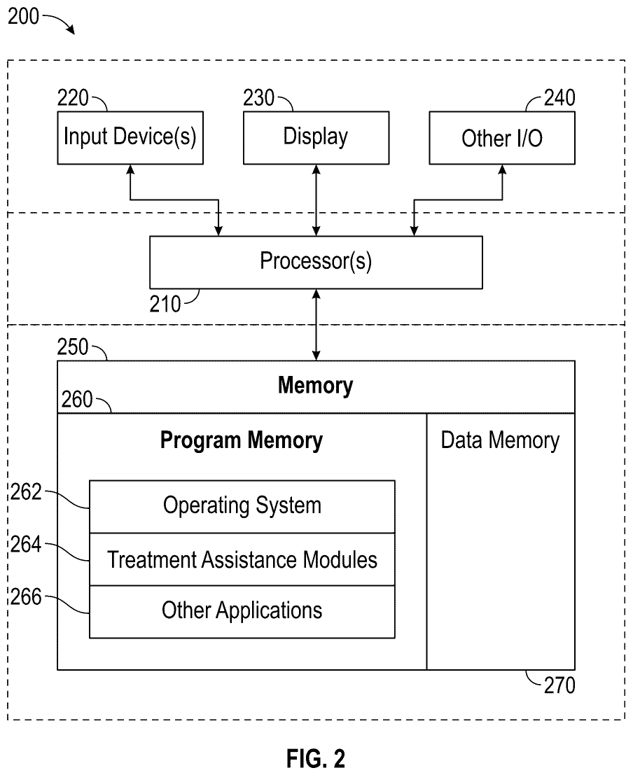

FIG. 2 illustrates a computing device 200 suitable for use in connection with the system 100 of FIG. 1, according to an embodiment. The computing device 200 can be incorporated in various components of the system 100 of FIG. 1, such as the client computing device 102 or the server 106. The computing device 200 includes one or more processors 210 (e.g., CPU(s), GPU(s), HPU(s), etc.). The processor(s) 210 can be a single processing unit or multiple processing units in a device or distributed across multiple devices. The processor(s) 210 can be coupled to other hardware devices, for example, with the use of a bus, such as a PCI bus or SCSI bus. The processor(s) 210 can be configured to execute one more computer-readable program instructions, such as program instructions to carry out of any of the methods described herein.

The computing device 200 can include one or more input devices 220 that provide input to the processor(s) 210, e.g., to notify it of actions from a user of the device 200. The actions can be mediated by a hardware controller that interprets the signals received from the input device and communicates the information to the processor(s) 210 using a communication protocol. Input device(s) 220 can include, for example, a mouse, a keyboard, a touchscreen, an infrared sensor, a touchpad, a wearable input device, a camera- or image-based input device, a microphone, or other user input devices.

The computing device 200 can include a display 230 used to display various types of output, such as text, models, virtual procedures, surgical plans, implants, graphics, and/or images (e.g., images with voxels indicating radiodensity units or Hounsfield units representing the density of the tissue at a location). In some embodiments, the display 230 provides graphical and textual visual feedback to a user. The processor(s) 210 can communicate with the display 230 via a hardware controller for devices. In some embodiments, the display 230 includes the input device(s) 220 as part of the display 230, such as when the input device(s) 220 include a touchscreen or is equipped with an eye direction monitoring system. In alternative embodiments, the display 230 is separate from the input device(s) 220. Examples of display devices include an LCD display screen, an LED display screen, a projected, holographic, or augmented reality display (e.g., a heads-up display device or a head-mounted device), and so on.

Optionally, other I/O devices 240 can also be coupled to the processor(s) 210, such as a network card, video card, audio card, USB, firewire or other external device, camera, printer, speakers, CD-ROM drive, DVD drive, disk drive, or Blu-Ray device. Other I/O devices 240 can also include input ports for information from directly connected medical equipment such as imaging apparatuses, including MRI machines, X-Ray machines, CT machines, etc. Other I/O devices 240 can further include input ports for receiving data from these types of machine from other sources, such as across a network or from previously captured data, for example, stored in a database.

In some embodiments, the computing device 200 also includes a communication device (not shown) capable of communicating wirelessly or wire-based with a network node. The communication device can communicate with another device or a server through a network using, for example, TCP/IP protocols. The computing device 200 can utilize the communication device to distribute operations across multiple network devices, including imaging equipment, manufacturing equipment, etc.

The computing device 200 can include memory 250, which can be in a single device or distributed across multiple devices. Memory 250 includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. In some embodiments, the memory 250 is a non-transitory computer-readable storage medium that stores, for example, programs, software, data, or the like. In some embodiments, memory 250 can include program memory 260 that stores programs and software, such as an operating system 262, one or more treatment assistance modules 264, and other application programs 266. The treatment assistance module(s) 264 can include one or more modules configured to perform the various methods described herein (e.g., the data analysis module 116 and/or treatment planning module 118 described with respect to FIG. 1). Memory 250 can also include data memory 270 that can include, e.g., reference data, configuration data, settings, user options or preferences, etc., which can be provided to the program memory 260 or any other element of the computing device 200.

### B. Select Methods of Modeling and Designing Patient-Specific Surgical Plans and Associated Patient-Specific Implants

The present technology includes systems and methods for designing and/or generating one or more patient-specific surgical plans and associated patient specific implants. In some embodiments, the patient-specific surgical plan is for a spinal fusion surgery, and the patient-specific implant is a patient-specific fusion device. For example, the spinal fusions surgery can include cervical fusion procedure, and the patient-specific implant can be a patient-specific cervical interbody implant.

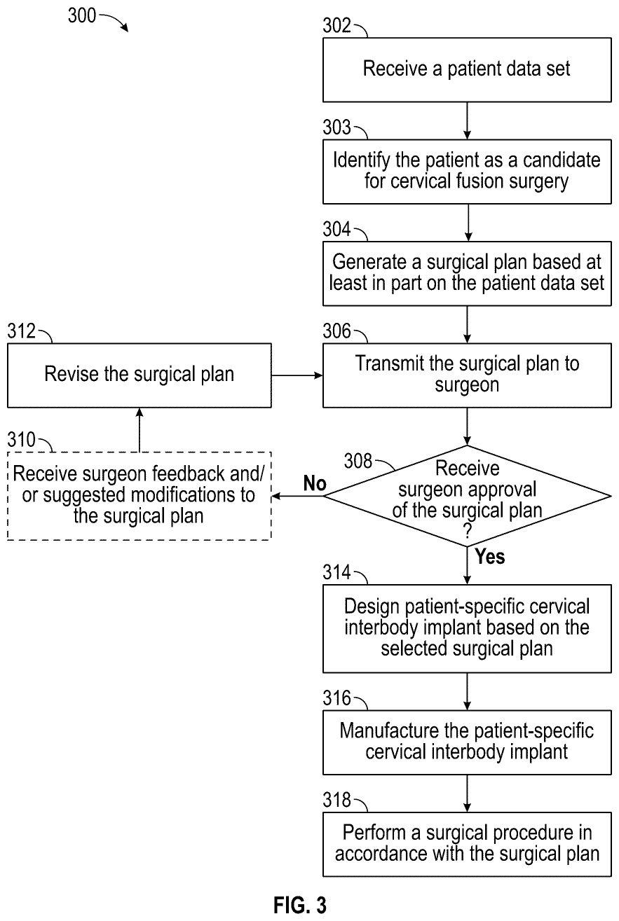

FIG. 3 is a flow diagram illustrating a method 300 for providing patient-specific medical care, according to an embodiment of the present technology. Some or all of the method 300 can be performed by various computing systems or software modules, including, for example, the computing systems described above with respect to FIGS. 1 and 2.

The method 300 can begin at block 302 by receiving a patient data set for a particular patient in need of medical treatment. The patient data set can include data representative of the patient's condition, anatomy, pathology, symptoms, medical history, preferences, and/or any other information or parameters relevant to the patient. For example, the patient data set can include surgical intervention data, treatment outcome data, progress data (e.g., surgeon notes), patient feedback (e.g., feedback acquired using quality of life questionnaires, surveys), clinical data, patient information (e.g., demographics, sex, age, height, weight, type of pathology, occupation, activity level, tissue information, health rating, comorbidities, health related quality of life (HRQL)), vital signs, diagnostic results, medication information, allergies, diagnostic equipment information (e.g., manufacturer, model number, specifications, user-selected settings/configurations, etc.) or the like. The patient data set can also include image data, such as camera images, Magnetic Resonance Imaging (MRI) images, ultrasound images, Computerized Aided Tomography (CAT) scan images, Positron Emission Tomography (PET) images, X-Ray images, and the like. In some embodiments, the patient data set includes data representing one or more of patient identification number (ID), age, gender, body mass index (BMI), lumbar lordosis, Cobb angle(s), pelvic incidence, disc height, vertebral body height, segment flexibility, bone quality, rotational displacement, and/or treatment level of the spine. The patient data set can be received at a server, computing device, or other computing system. For example, in some embodiments the patient data set can be received by the server 106 shown in FIG. 1. In some embodiments, the computing system that receives the patient data set at block 302 also stores one or more software modules (e.g., the data analysis module 116, the treatment planning module 118, the disease progression module 120, and/or the intervention timing module 121, shown in FIG. 1, or additional software modules for performing various operations of the method 300).

In some embodiments, the received patient data set can include disease metrics such as lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.), cervical parameters, thoracic parameters, lumbar parameters, and/or pelvic parameters. The disease metrics can include micro-measurements (e.g., metrics associated with specific or individual segments of the patient's spine) and/or macro-measurements (e.g., metrics associated with multiple segments of the patient's spine). In some embodiments, the disease metrics are not included in the patient data set, and the method 300 includes determining (e.g., automatically determining) one or more of the disease metrics based on the patient image data, as described below. In some embodiments, the received patient data can include functional mobility test scores (e.g., step test, six-meter walk test, sit-to-stand test, timed up and go test, etc.). The received patient data set can include additional subjective test scores that reflect aspects of the patient condition, such as pain tests (e.g., Visual Analog Scale (VAS) pain scores, Low Back Pain Rating scale scores, etc.), disability tests (e.g., Oswestry Disability Index scores, Quebec back pain disability test scores, etc.), quality of life tests (e.g., Quality of Life Scale scores), etc.

The method 300 can continue at block 303 by identifying the patient as a candidate for spinal fusion surgery. In some embodiments, the operation at block 303 includes analyzing the received patient data set from the operation in block 302 to determine whether the patient would benefit from spinal fusion surgery. In some embodiments, the operation of identifying the patient as a candidate for spinal fusion surgery can be performed by one or more treatment planning programs or modules, such as described with reference to FIG. 1.

If the patient is identified as being a candidate for spinal fusion surgery, the method 300 can continue at block 304 by generating a surgical plan based at least in part on the patient data set received at block 302. As described in detail below, the surgical plan can include a target location or region of interest for surgical intervention and one or more surgical procedures or interventions to be performed at the region of interest. The surgical plan can also include predicted post-operative data associated with performing the surgical procedure at the target location. For example, the surgical plan may include a predicted or target post-operative anatomical configuration shown as a two or three dimensional virtual model. In some embodiments, the surgical plan also includes additional predicted post-operative analytics, such as predicted disease progression, predicted patient satisfaction, predicted patient mobility, predicted patient pain, predicted patient quality of life, etc.

In some embodiments, the operation of generating the surgical plan includes identifying a specific target location to be involved in the surgical procedure. For example, in the context of spinal fusion surgery, generating the surgical plan may include identifying one or more vertebral levels for fusion. In some embodiments, the vertebral level is a cervical vertebral level (e.g., C1-C7). In some embodiments, the identified target location includes a specific range of vertebral levels to be involved in a surgery (e.g., C3-C4, C4-C6, etc.). The identified target location may include two, three, four, five, or more vertebral levels. Of course, the foregoing target locations are provided by way of example only, and the present technology is not limited to the anatomical locations listed above. Indeed, in some embodiments the target location may include other vertebral levels, such as lumbar and/or thoracic vertebral levels, and/or anatomical structures other than the spine, such as the hip, knee, ankle, shoulder, elbow, wrist, hand, the jaw, the skull, or other anatomical locations, as described throughout this Detailed Description.

The target location can be identified by reviewing image data of the patient. In some embodiments, a computing system (e.g., the server 106 of FIG. 1) and/or one or more software modules (e.g., the treatment planning module 118 of FIG. 1) can review and analyze patient image data and automatically identify the target location. In such embodiments, a trained machine learning program or other software-based program can analyze patient image data, extract measurements from the patient image data, compare the extracted measurements to reference data (e.g., predetermined thresholds or ranges associated with “healthy” patients normalized for age, sex, gender, etc.), and identify anatomical regions that are candidates for surgical correction. Alternatively or additionally, the target location can be identified and/or confirmed through other suitable means, such as via a technician or healthcare provider reviewing image data and identifying anatomical deformities.

As provided above, in some embodiments the operation of generating the surgical plan also includes identifying a surgical procedure for the patient. In embodiments in which the surgical plan includes identifying a target location, the surgical procedure can be associated with the target location. In the context of spinal surgery, representative surgical procedures include spinal fusion, artificial disc replacement, vertebroplasty, kyphoplasty, spinal laminectomy/decompression, discectomy, facetectomy, foraminotomy, or other spine surgery procedures. Examples of spinal fusion surgery include as anterior cervical fusion (ACF), posterior cervical fusion (PCF), posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). The foregoing are provided by way of example only, and the present technology can include identifying any type of spinal or other surgical procedures at block 304.

The surgical procedure associated with the surgical plan can be identified using any of the methods and systems described herein. For example, in some embodiments the server 106 of FIG. 1 and/or the associated software modules (e.g., the treatment planning module 118) can identify one or more surgical procedures based on, for example, user input, the received or extracted patient data, and/or the identified target location(s). For example, if the server 106 determines that a patient is suffering from disc degeneration between the C3 and C4 vertebral bodies, the server 106 may recommend an ACF procedure to fuse C3 and C4. Alternatively, the server 106 may recommend an artificial disc replacement between C3 and C4 to correct the degeneration while preserving motion. The surgical procedure can be identified using other methods and systems as well.

In some embodiments, the operation at block 304 can include reviewing and/or analyzing multiple types of surgical procedures and/or surgical steps to identify the surgical procedure for inclusion within the surgical plan. Types of surgical procedures and/or surgical steps can be selected for inclusion with the surgical plan (or eliminated from inclusion with the surgical plan) based on, for example, user input, insurance coverage of the procedure or step, healthcare provider parameters (e.g., based on healthcare provider ranking/scores such as hospital/physician expertise, number of similar procedures performed, hospital ranking for procedure, etc.), healthcare resource parameters (e.g., diagnostic equipment, facilities, surgical equipment such as surgical robots), and/or other non-patient related information (e.g., information that can be used to score, predict outcomes and risk profiles for procedures for the present healthcare provider, and/or rank procedures).

In some embodiments, the operation of generating the surgical plan includes identifying or designing a corrected anatomical configuration for the patient (the corrected anatomical configuration can also be referred to herein as the “planned configuration,” “optimized geometry,” “post-operative anatomical configuration,” or “target outcome”). The corrected anatomical configuration can reflect the desired and/or predicted anatomy of the patient if the surgical plan were performed. In some embodiments, generating the surgical plan includes generating one or more virtual models (two-dimensional models, three-dimensional models, etc.) showing the corrected anatomical configuration. The virtual model may include some or all of the patient's anatomy within the target location (e.g., any combination of tissue types including, but not limited to, bony structures, cartilage, soft tissue, vascular tissue, nervous tissue, etc.). In some embodiments, the corrected anatomical configuration is identified/determined before the surgical procedure and/or target location. That is, a computing system or user can model a preferred anatomical outcome, and, based on the desired anatomical outcome, identify a surgical procedure and target location that will achieve the desired anatomical outcome once performed.

In some embodiments, generating the surgical plan includes generating one or more patient metrics associated with the corrected anatomical configuration. In the context of spinal surgery, patient metrics may include, for example, coronal parameters, sagittal parameters, pelvic parameters, Cobb angles, shoulder tilt, iliolumbar angles, coronal balance, lordosis angles, intervertebral space height, vertebral endplate coverage, or other similar spinal parameters. Similar as described above, the patient metrics can be determined before identifying a surgical procedure and/or target location for surgical intervention. That is, a computing system or user can use the patient metrics to identify a surgical procedure and target location that will achieve the patient metrics once performed.

The surgical plan can include additional features. In some embodiments, for example, the surgical plan can include predicted disease progression, predicted patient satisfaction, predicted patient mobility, predicted patient pain, predicted patient quality of life, or the like. For example, the surgical plan may include estimates of disease progression if the patient were to undergo the identified surgical procedure at the identified target location. That is, the surgical plan can include virtual models (e.g., two-dimensional or three-dimensional virtual models) of patient anatomy at various intervals post-operation. For example, the surgical plan may include a predictive model of patient anatomy at one or more of 6 months post-op, 1 year post-op, 2 years post-op, 3 years post-op, 4 year post-op, 5 years post-op, 6 years post-op, 7 years post-op, 8 years post-op, 9 years post-op, and/or 10 years post-op. The disease progression model may also include predicted patient metrics (e.g., any of the patient metrics described herein, including coronal parameters, sagittal parameters, pelvic parameters, Cobb angles, shoulder tilt, iliolumbar angles, coronal balance, lordosis angles, intervertebral space height, or other similar spinal parameters) at any of the various post-operative intervals identified above, in addition to or in lieu of including the virtual model of predicted patient anatomy.

Once generated, the surgical plan can be digitally displayed as a surgical report on one or more display screens for ease of review, editing, annotation, the like. In some embodiments, the surgical plan can be stored as computer-executable instructions that can be executed via the surgical plan review module 123 on the client computing device 102 of FIG. 1.

In some embodiments, the operation of generating the surgical plan at block 304 includes generating a plurality of candidate surgical plans (or subsets of surgical plans such as surgical procedures), and then selecting the surgical from within the plurality of candidate surgical plans. For example, in some embodiments a computing system can automatically identify a plurality (e.g., two, three, four, five, six, seven, eight, nine, ten, or more) of surgical plans (or subsets of surgical plans such as surgical procedures) based on the patient-data set and/or one or more user-inputted criteria. The identified candidate surgical plans may be ranked and/or scored based on various factors, including predicted patient outcomes, user-review, etc. The highest ranked identified candidate surgical plans (e.g., based on predicted patient outcomes) can be selected as the surgical plan. In some embodiments, certain ranked surgical plans may not be selected as the surgical plan based on user review and/or failure to meet various user criteria. For example, if a particular surgical plan is identified as requiring a surgical procedure that the physician is unfamiliar with, the particular surgical plan may not be selected, and the method can instead include selecting the next best surgical plan as the surgical plan. Accordingly, in some implementations, physician-specific scoring is used to score candidate procedures/surgical plans before selecting the surgical plan. For example, procedures with scores meeting a threshold score (e.g., threshold post-operative metrics score, physician inputted threshold score, threshold outcome score, etc.) can be identified for user review. The system can therefore compare advantages and disadvantages of candidate procedures with respect to each other before selecting the surgical plan. Additional features of generating and comparing multiple surgical plans are described in U.S. patent application Ser. No. 18/455,881, the disclosure of which is incorporated by reference herein in its entirety.

Once the surgical plan is generated at block 304, the method 300 can continue at block 306 by transmitting the surgical plan to a surgeon. In some embodiments, the same computing system used at blocks 302 and 304 can transmit the surgical plan to a computing device for surgeon review (e.g., the client computing device 102 described in FIG. 1). This can include directly transmitting the surgical plan to the computing device or uploading the first and second surgical plans to a cloud or other storage system for subsequent downloading.

The surgeon can review the surgical plan and, at block 308, approve or disapprove of the surgical plan. For example, the surgeon may review the surgical plan using the surgical plan reviewing program 125 (FIG. 1) to determine whether the surgeon deems the surgical plan acceptable. This may include, for example, reviewing the surgical plans target locations, surgical procedure, target/predicted post-operative anatomical configuration, and predicted post-operative patient metrics.

In some embodiments, the surgeon may not approve the surgical plan at block 308. In such embodiments, the surgeon can optionally provide feedback and/or suggested modifications to the surgical plan (e.g., by adjusting the virtual model or changing one or more aspects about the plan, providing comments on or more requested changes to the surgical plan, etc.). Accordingly, the method 300 can optionally include receiving (e.g., via the computing system) the surgeon feedback and/or suggested modifications at block 310. This may include, for example, modifying target locations for surgical intervention, surgical procedures, and/or target post-operative anatomical configuration. If surgeon feedback and/or suggested modifications are received at block 310, the method 300 can continue at block 312 by revising (e.g., automatically revising via the computing system) the surgical plan based at least in part on the surgeon feedback and/or suggested modifications received at block 310. In some embodiments, the surgeon does not provide feedback and/or suggested modifications if they reject the surgical plan. In such embodiments, block 310 can be omitted, and the method 300 can continue at block 312 by revising (e.g., automatically revising via the computing system) the surgical plans by selecting new and/or additional reference patient data sets and/or generating a new candidate surgical plan. The revised and/or new surgical plan can then be transmitted to the surgeon for review. The operations at blocks 306, 308, 310, and 312 can be repeated as many times as necessary until the surgeon selects and approves a particular surgical plan.

Once surgeon approval of a surgical plan is received at block 308, the method 300 can continue at block 314 by designing (e.g., via the same computing system that performed blocks 302-308) a patient-specific fusion implant based on the selected surgical plan. For example, the patient-specific fusion implant can be designed based on the target location and surgical procedure included in the selected surgical plan. The patient-specific implant(s) can also be specifically designed such that, when implanted in the particular patient at the target location using the identified surgical procedure, it directs the patient's anatomy to occupy the target post-operative anatomical configuration (e.g., transforming the patient's anatomy from the patient's native anatomical configuration to the corrected anatomical configuration). The patient-specific fusion implant can be designed such that, when implanted, it causes the patient's anatomy to occupy the corrected anatomical configuration for the expected service life of the implant (e.g., 5 years or more, 10 years or more, 20 years or more, 50 years or more, etc.). In some embodiments, the patient-specific fusion implant is designed solely based on the virtual model of the corrected anatomical configuration and/or without reference to pre-operative patient images.

The patient-specific fusion implant can be any of the implants described herein. For example, the patient-specific fusion implant can be any of the cervical fusion interbody implants described in Section C of this Detailed Description. In other embodiments, the patient-specific fusion implant can include other implants, such as those described in U.S. application Ser. Nos. 16/048,167, 16/242,877, 16/207,116, 16/352,699, 16/383,215, 16/569,494, 16/699,447, 16/735,222, 16/987,113, 16/990,810, 17/085,564, 17/100,396, 17/342,329, 17/518,524, 17/531,417, 17/835,777, 17/851,487, 17/867,621, and 17/842,242 and International Patent Application No. PCT/US2024/010202, each of which is incorporated by reference herein in its entirety. The patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include implant shape, size, material, and/or effective stiffness (e.g., lattice density, number of struts, location of struts, etc.). In addition to the interbody device, in some embodiments the patient-specific fusion implant can further include one or more screws (e.g., bone screws, spinal screws, pedicle screws, facet screws), cages, plates, rods, discs, spacers, expandable devices, stents, brackets, ties, scaffolds, fixation device, anchors, nuts, bolts, rivets, connectors, tethers, fasteners, or the like.

In some embodiments, designing the implant at block 316 can optionally include generating fabrication instructions for manufacturing the implant. For example, the computing system may generate computer-executable fabrication instructions that that, when executed by a manufacturing system, cause the manufacturing system to manufacture the implant.

In some embodiments, the patient-specific implant is designed at block 316 only after the surgeon has selected a surgical plan. Accordingly, in some embodiments, the implant design is neither transmitted to the surgeon with the surgical plan at block 308, nor manufactured before receiving surgeon approval of the surgical plan. Without being bound by theory, waiting to design the patient-specific implant until after the surgeon approves the surgical plan may increase the efficiency of the method 300 and/or reduce the resources necessary to perform the method 300. In other embodiments, one or more patient-specific implants can be designed and included in the surgical plans transmitted to the surgeon at block 306. For example, a virtual implant of the patient-specific fusion implant can be generated and transmitted for surgeon review concurrent with the surgical plan during the operation of block 306. Accordingly, in some embodiments the operation at block 314 can be included within the block 304.

The method 300 can continue at block 316 by manufacturing the patient-specific fusion implant. The implant can be manufactured using additive manufacturing techniques, such as 3D printing, stereolithography, digital light processing, fused deposition modeling, selective laser sintering, selective laser melting, electronic beam melting, laminated object manufacturing, powder bed printing, thermoplastic printing, direct material deposition, or inkjet photo resin printing, or like technologies, or combination thereof. Alternatively or additionally, the implant can be manufactured using subtractive manufacturing techniques, such as CNC machining, electrical discharge machining (EDM), grinding, laser cutting, water jet machining, manual machining (e.g., milling, lathe/turning), or like technologies, or combinations thereof. The implant may be manufactured by any suitable manufacturing system (e.g., the manufacturing system 124 shown in FIG. 1). In some embodiments, the implant is manufactured by the manufacturing system executing the computer-readable fabrication instructions generated by the computing system at block 316.

Once the implant is manufactured at block 316, the method 300 can continue at block 318 by performing the selected surgical plan and implanting the patient-specific fusion implant into the patient. Aspects of the surgical plan, such as some or all of the surgical procedure, can be performed manually, by a robotic surgical platform (e.g., a surgical robot), or a combination thereof. In embodiments in which the surgical procedure is performed at least in part by a robotic surgical platform, the surgical plan can include computer-readable control instructions configured to cause the surgical robot to perform, at least partly, the patient-specific surgical procedure.

The method 300 can be implemented and performed in various ways. In some embodiments, the operations at blocks 302-314 can be performed by a computing system associated with a first entity, block 316 can be performed by a manufacturing system associated with a second entity, and block 318 can be performed by a surgical provider, surgeon, and/or robotic surgical platform associated with a third entity. Any of the foregoing blocks may also be implemented as computer-readable instructions stored in memory and executable by one or more processors of the associated computing system(s).

### C. Select Embodiments of Patient-Specific Spinal Fusion Devices

The systems and methods described with reference to FIGS. 1-3 can be used to design and manufacture patient-specific medical devices for use with a patient-specific surgical plan. In some embodiments, the patient-specific medical devices include a patient-specific spinal fusion device. The patient-specific spinal fusion devices can include one or more interbody implants for spinal fusion surgery, such as ACF implants, PCF implants, PLIF implants, ALIF implants, TLIF implants, LLIF implants, DLIF implants, and/or XLIF implants. In some embodiments, the patient-specific fusion devices further include, in addition to the interbody device, one or more fixation elements or screws configured to anchor the interbody device to patient anatomy. Accordingly, in some embodiments the patient-specific spinal fusion devices include at least a patient-specific interbody device and one or more screws for fixating the patient-specific interbody devices to patient anatomy.

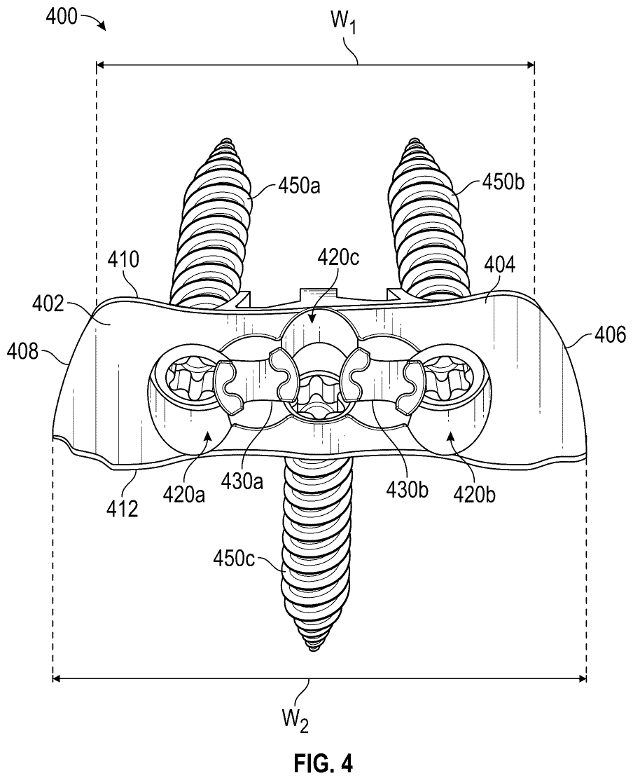

FIG. 4 is a front view of a representative patient-specific spinal fusion device 400 (“the device 400”) configured in accordance with select embodiments of the present technology. The device 400 can include a patient-specific interbody implant 402 (which can also be referred to herein as an intervertebral spacer, implant body, or the like) and a plurality of fixation elements or screws 450 (shown individually as a first fixation element 450a, a second fixation element 450b, and a third fixation element 450c). As described in greater detail below, the implant 402 can be sized and shaped to be positioned within a disc space (e.g., after a discectomy) between adjacent vertebral bodies to promote fusion of two or more vertebral bodies. The fixation elements 450 can include screws, anchors, bolts, or other suitable components for securing the interbody implant 402 to the vertebral bodies.

The implant 402 has an anterior surface or face 404, a posterior surface or face (not visible in FIG. 4), a first lateral or side surface or face 406, a second lateral or side surface or face 408, a superior surface or face 410, and an inferior surface or face 412. As described in greater detail below, the superior surface 410 can be designed to contact (e.g., mate with) an inferior surface of a superior vertebral body, and the inferior surface 412 can be designed to contact (e.g., mate with) a superior surface of an inferior vertebral body. Accordingly, the superior surface 410 can also be referred to as a superior endplate 410 of the implant 402, and the inferior surface can also be referred to as an inferior endplate 412 of the implant 402. As described in greater detail below, the superior endplate 410 and the inferior endplate 412 can have different sizes and/or dimensions.

The implant 402 further includes a first lumen 420a, a second lumen 420b, and a third lumen 420c (collectively referred to as lumens 420). The lumens 420 can be bore, screw, or anchor holes or channels that are configured to receive the fixation elements 450. Accordingly, each of the lumens 420 can include a first (e.g., “entry”) aperture in the anterior surface 404 of the implant 402, and a second (e.g., “exit”) aperture. The exit aperture can be either in the superior endplate 410 or the inferior endplate 412, depending on the angular orientation of the corresponding lumen 420. For example, in the illustrated embodiment the first lumen 420a and the second lumen 420b have exit apertures in the superior surface 410, and the third lumen 420c has an exit aperture in the inferior surface 412. The angled orientation of the lumens 420 sets the angled orientation of the fixation elements 450, and thus can be designed based on a desired fixation angle and/or desired fixation target.

In some embodiments, the implant 402 can include more or fewer lumens 420, e.g., such that the device 400 includes more or fewer fixation elements 450, such as one, two, four, five, six, seven, or more fixation elements 450. Similarly, the lumens 420 (and therefore the fixation elements 450) can have different orientations than those shown in FIG. 4. For example, in some embodiments the implant 402 can include one lumen 420 (and therefore one fixation element 450) having a superior-posterior angle and two lumens 420 (and therefore two fixation elements 450) having an inferior-posterior angle. As another example, the implant 400 can include one lumen 420 having a superior-posterior angle and one lumen 420 having an inferior-posterior angle.

In some embodiments, an inner surface of the lumens 420 is smooth, as opposed to rough and/or textured. In such embodiments, the inner surface of the lumens 420 can be smoothed during a specific step during manufacture of the implant 402, although in other embodiments the inner surface is relatively smooth as a result of the manufacturing process used to produce the implant 402, without the need for an additional step to smooth the inner surface. Without intending to be bound by theory, having a relatively smooth inner surface for the lumens 420 is expected to be advantageous because it reduces friction between the fixation elements 450 and the inner surface, e.g., as the fixation elements 450 are advanced through the lumens 420 and anchored to patient anatomy. In some embodiments, the inner surfaces of the lumens 420 can include a thread for engaging the fixation elements 450, even in embodiments in which the surface is otherwise smooth. In some embodiments, the inner surface is a continuous surface that extends (e.g., without gaps or apertures) from the entry aperture of each lumen 420 to the exit aperture of each lumen 420.

The implant 402 further includes a first retention mechanism 430a and a second retention mechanism 430b (collectively referred to as “the retention mechanisms 430”) for retaining the fixation elements 450 within the lumens 420. The retention mechanisms 430 can be selectively rotated or otherwise manipulated between a first, unlocked configuration (not shown in FIG. 4) that permits a user to insert the fixation elements 450 into the corresponding lumens 420, and a second, locked configuration (illustrated in FIG. 4) that locks the fixation elements 450 in the corresponding lumens 420. For example, the retention mechanisms 430 can each include a rotatable cam or tab that in the unlocked configuration does not overlap with or block the corresponding lumens 420, and in the locked configuration does at least partially overlap and/or block the corresponding lumens. In particular, in the locked configuration, the first retention mechanism 430a can hold (e.g., prevent back-out of) the first fixation element 450a and the third fixation element 450c in the first lumen 510a and the third lumen 510c, respectively, and the second retention mechanism 430b can hold the second fixation element 450b and the third fixation element 450c in the second lumen 420b and the third lumen 420c, respectively. Additional details of retention mechanisms that can be used with the implant 402 are described in International Patent Application Publication No. WO2024/148108, the disclosure of which is incorporated by reference herein in its entirety.

In some embodiments, the implant 402 is a single, contiguous component (e.g., a one-piece interbody implant). For example, the implant 402 can be manufactured as a single structure using various additive manufacturing techniques. In some embodiments, the implant 402 is composed of metal (e.g., titanium, etc.) and/or a metal alloy (e.g., stainless steel, Nitinol, etc.). In other embodiments, the implant 402 can be composed of a biocompatible plastic. The implant 402 can also include a combination of lattice portions and solid portions. The combination of lattice portions and solid portions can be designed based on desired implant properties, such as stiffness, load-bearing capabilities, promotion of bone growth, fit, cost, or the like. In some embodiments, the implant 402 is expandable between a low-profile delivery configuration and a deployed configuration, such as described in U.S. Patent Application Publication No. 2022/0387191, the disclosure of which is incorporated by reference herein in its entirety.

The implant 402 can have one or more “patient-specific” features designed to correspond to a particular patient's anatomy. Accordingly, in some embodiments the device 400 can be designed and manufactured using the systems described with reference to FIGS. 1 and 2 and/or the method described with reference to FIG. 3. In other embodiments, the device 400 can be manufactured using other suitable systems and methods that incorporate patient-specific parameters into the design of the device 400.

For example, one or more surfaces of the implant can be designed to have a topography that matches (e.g., mates with) a topography of patient anatomy that the implant surface will contact once the implant 402 is implanted in the patient. For example, in embodiments in which the implant 402 is configured for placement in the C2-C3 disc space, the superior endplate 410 of the implant 402 can have a topography configured to mate with a topography of the inferior endplate of the C2 vertebral body, and the inferior endplate 412 of the implant 402 can have a topography configured to mate with a topography of the superior endplate of the C3 vertebral body. As a result, the superior endplate 410 and the inferior endplate 412 can be irregularly contoured to match a contouring of the adjacent vertebral endplates. Also as a result, the implant 402 can be asymmetrical with respect to a mid-sagittal plane of the implant 402 and/or with respect to a transverse plane of the implant 402. Other properties of the implant (e.g., size, geometry, load-bearing characteristics, shear forces, and any other properties that can be made patient-specific as described with reference to FIGS. 1-3) can also be patient-specific.

In addition to or in lieu of having patient-specific topographies, the size of the superior endplate 410 and the inferior endplate 412 can be patient-specific. In such embodiments, the superior endplate 410 and the inferior endplate 412 can have different sizes and dimensions. For example, the superior endplate 410 can have a first transverse diameter W₁ (e.g., width) and the inferior endplate 412 can have a second transverse diameter W₂ that is different than the first transverse diameter W₁. In the illustrated embodiment, the second transverse diameter W₂ of the inferior endplate 412 is greater than the first transverse diameter W₁ of the superior endplate 410. For example, the second transverse diameter W₂ of the inferior endplate 412 may be between about 2%-50% greater than, or between about 2%-25% greater than, or between about 5%-20% greater than, or between about 10%-15% greater than the first transverse diameter W₁ of the superior endplate 410. In such embodiments, the width of the anterior surface 404 increases in the superior to inferior direction. In other embodiments, the first transverse diameter W₁ of the superior endplate 410 is greater than the second transverse diameter W₂ of the inferior endplate 412. For example, the first transverse diameter W₁ of the superior endplate 410 may be between about 2%-50% greater than, or between about 2%-25% greater than, or between about 5%-20% greater than, or between about 10%-15% greater than the second transverse diameter W₂ of the inferior endplate 412. In such embodiments, the width of the anterior surface 404 increases in the inferior to superior direction.

The superior endplate 410 and the inferior endplate 412 can have other dimensions that differ, in addition to or in lieu of the transverse diameter (e.g., width). For example, the superior endplate 410 can have a first anteroposterior diameter (e.g., depth) and the inferior endplate 412 can have a second anteroposterior diameter that is different than the first anteroposterior diameter. The first anteroposterior diameter can be greater than the second anteroposterior diameter, e.g., by between about 2%-50%, about 2%-25%, about 5%-20%, or about 10%-15%. In other embodiments, the second anteroposterior diameter can be greater than the first anteroposterior diameter, e.g., by between about 2%-50%, about 2%-25%, about 5%-20%, or about 10%-15%.

The superior endplate 410 and the inferior endplate 412 can also have shapes that are patient-specific. For example, the superior endplate 410 and/or the inferior endplate 412 can have a circular, triangular, rectangular, oblong, or irregular shape designed based on the shape of the vertebral body the implant is configured to contact. In some embodiments, the shape of the superior endplate 410 and the inferior endplate 412 is different. For example, the superior endplate 410 may have a generally circular shape, and the inferior endplate 412 may have a generally rectangular shape. In other embodiments, the shape of the superior endplate 410 and the inferior endplate 412 are the same, but the sizes differ. For example, both the superior endplate 410 and the inferior endplate 412 may have a generally circular shape.

As a result, the superior endplate 410 and the inferior endplate 412 can have different sizes and/or surface areas. Further, the cross-sectional area of the implant 402 can be variable. In some embodiments, for example, the cross-sectional area of the implant 402 can increase in the superior to inferior direction. In other embodiments, the cross-sectional area of the implant 402 can decrease in the superior to inferior direction. The change in cross-sectional area between the superior endplate 410 and the inferior endplate 412 can be linear, curved, or irregular. As described in greater detail below, and without intending to be bound by theory, providing implants having superior and inferior endplates with different dimensions is expected to enable the implants to have better coverage of both of the corresponding vertebral body endplates. In turn, this is expected to provide better patient outcomes, such as faster or more robust fusion, greater stability, and/or decreased likelihood of implant subsidence, expulsion, or other side effects.

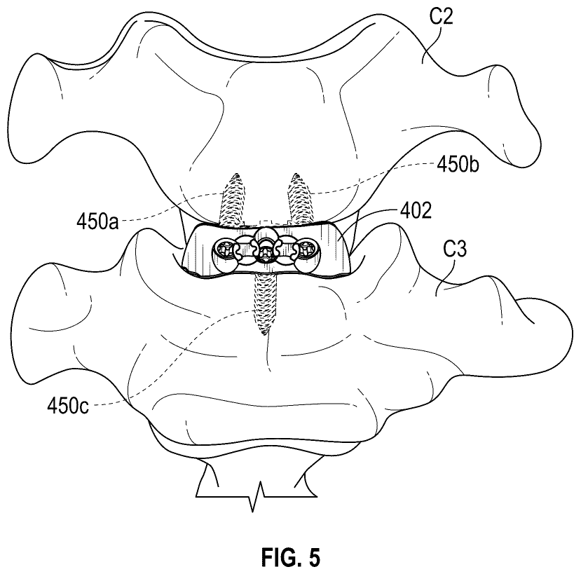

FIG. 5 illustrates the device 400 including the implant 402 and the fixation elements 450 implanted in a patient's spine within the disc space between the C2 and C3 vertebral bodies in accordance with embodiments of the present technology. Although shown as being implanted between the C2 and C3 vertebral bodies, one skilled in the art will appreciate that the device 400 can be implanted between other cervical vertebral levels, or more generally at other portions of the patient's spine, including the thoracic, lumbar, and sacral regions. In the illustrated embodiment, the implant 402 is secured to the C2 and C3 vertebral bodies using the fixation elements 450. In particular, the first fixation element 450a and the second fixation element 450b are angled superiorly and posteriorly and thus extend in to the C2 vertebral body, and the third fixation element 450c is angled inferiorly and posteriorly and thus extends into the C3 vertebral body. Without intending to be bound by theory, anchoring the interbody implant 402 to both the superior and inferior vertebrae is expected to reduce the risk of implant expulsion from the disc space prior to fusion.

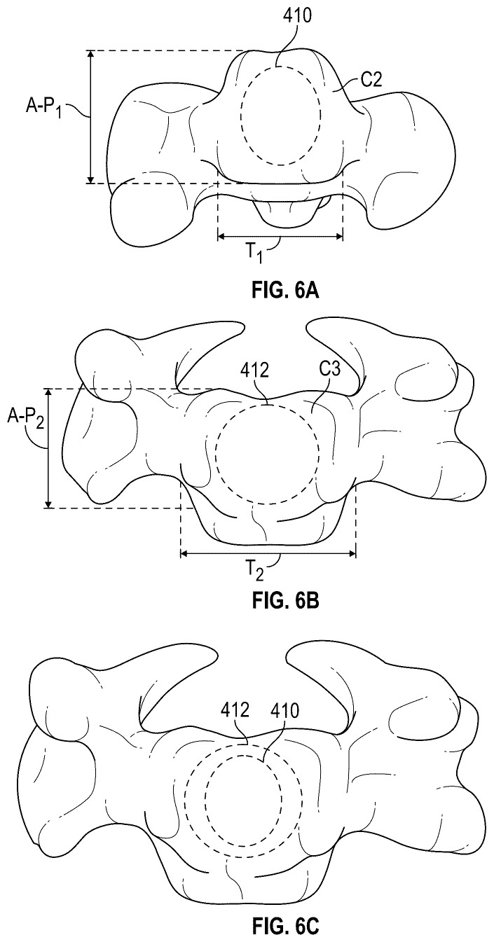

The size of the superior endplate 410 and the inferior endplate 412 can be designed based on the corresponding size of the vertebral endplates they will contact once implanted in the patient. Different vertebral bodies have different shapes and sizes. For example, FIG. 6A is a bottom view of the C2 vertebral body from FIG. 5 showing the inferior endplate of the C2 vertebral body, and FIG. 6B is a top view of the C3 vertebral body from FIG. 5 showing the superior endplate of the C3 vertebral body. Both FIGS. 6A and 6B omit the device 400 for purposes of clarity. As shown in FIG. 6A, the inferior endplate of the C2 vertebral body has a first transverse length T1 and a first anterior-posterior length AP1. As shown in FIG. 6B, the superior endplate of the C3 vertebral body has a second transverse length T2 and a second anterior-posterior length AP2. In a typical patient, the first transverse length T1 is different than the second transverse length T2, and the first anterior-posterior length AP1 is different than the second anterior-posterior length AP2. This is both because these distances are on different vertebral bodies (e.g., C2 versus C3) and different ends of the corresponding vertebral bodies (e.g., inferior endplate versus superior endplate).

As shown in broken line in FIG. 6A, the superior endplate 410 can be designed to have a footprint that covers at least 75%, or at least 80%, or at least 85%, or at least 90%, or at least 91%, or at least 92%, or at least 93%, or at least 94%, or at least 95% of the inferior vertebral endplate that it is designed to mate with. As used herein, “covering at least X %” refers to the overall footprint defined by an outer perimeter of the superior endplate 410 covering a percentage of an overall footprint defined by an outer perimeter of the vertebral endplate, and does not account for microcavities, micro-protrusions, or other features that may increase a total surface area of the superior endplate 410 and/or inferior vertebral endplate. Similarly, as shown in broken line in FIG. 6B, the inferior endplate 412 can be designed such that it covers at least 75%, or at least 80%, or at least 85%, or at least 90%, or at least 91%, or at least 92%, or at least 93%, or at least 94%, or at least 95% of the superior vertebral endplate that it is designed to mate with. In some embodiments, the superior endplate 410 and the inferior endplate 412 are designed to cover the same or at least about the same percent of the corresponding vertebral endplates they are configured to contact when the implant 402 is implanted in the patient. In other embodiments, the superior endplate 410 and the inferior endplate 412 are designed to cover a different percent of the corresponding vertebral endplates they are configured to contact when the implant 402 is implanted in the patient. For example, the superior endplate 410 can be designed to cover at least 5%, or at least 10%, or at least 15% more of the inferior vertebral endplate as compared to the percentage of the superior vertebral endplate that the inferior endplate 412 is designed to cover, or vice versa.

Regardless of whether the superior endplate 410 and inferior endplate 412 are designed to cover the same or different percent of the corresponding vertebral endplate, the vertebral endplates may have different sizes and/or surface areas. Indeed, while the superior endplate 410 and the inferior endplate 412 can be designed to cover the same percentage of the corresponding vertebral endplates (e.g., about 90%), the superior endplate 410 and the inferior endplate 412 would necessarily have different dimensions in order to do so. FIG. 6C illustrates representative footprints of the superior endplate 410 and the inferior endplate 412. As shown, the superior endplate 410 has an overall smaller footprint than the inferior endplate 412 due to the smaller dimensions of the inferior endplate of the C2 vertebral body (FIG. 6A) relative to the superior endplate of the C3 vertebral body (FIG. 6B).

In some embodiments, the superior endplate 410 and/or the inferior endplate 412 can be designed such that, when implanted, one or more sides or edges of the implant sit within a predetermined distance of a margin of the vertebral endplates. For example, in some embodiments the superior endplate 410 is sized and shaped such that, when the implant 402 is implanted at its target position, a distance between (a) the boundary between the anterior surface 404 of the implant 402 and the superior endplate 410 of the implant 402, and (b) an anterior margin of the inferior endplate of the superior vertebral body, is less than about 3.5 mm, less than about 3 mm, less than about 2.5 mm, less than about 2.0 mm, and/or less than about 1.5 mm. Similarly, a distance between (a) the boundary between the posterior surface of the implant 402 and the superior endplate 410 of the implant 402, and (b) a posterior margin of the inferior endplate of the superior vertebral body, is less than about 3.5 mm, less than about 3 mm, less than about 2.5 mm, less than about 2.0 mm, and/or less than about 1.5 mm. Still further, a distance between (a) the boundary between a lateral surface of the implant 402 and the superior endplate 410 of the implant 402, and (b) a lateral margin of the inferior endplate of the superior vertebral body, is less than about 3.5 mm, less than about 3 mm, less than about 2.5 mm, less than about 2.0 mm, and/or less than about 1.5 mm. Although described with respect to the distance between the vertebral endplate margins of the superior vertebral body and the superior endplate 410, the inferior endplate 412 can also be designed to reside within any of the foregoing dimensions of the margins of the superior endplate of the inferior vertebral body.

In some embodiments, the size and/or shape of the superior endplate 410 and/or the inferior endplate 412 is designed based on certain anatomical structures or regions of the corresponding vertebral body endplates. For example, in some embodiments the superior endplate 410 can be designed to overlap/contact a portion of the cortical rim of the corresponding vertebral endplate. The superior endplate 410 can overlap/contact the cortical rim at one, two, three, or four different margins (e.g., anterior, posterior, lateral, etc.). In some embodiments, the contact region at the cortical rim can be designed as a load-bearing portion of the implant 400. As another example, in some embodiments the superior endplate 410 can be designed to contact a threshold load-bearing amount of a central region of the corresponding vertebral body endplate and to be surrounded by the cortical rim. In such embodiments, a load bearing capability of the vertebral body can be determined (e.g., using FEA analysis, fracture analysis, stress analysis, simulations based on virtual models and patient information, etc.), and the threshold load-bearing amount of the central region can be determined based on the load bearing capability of the vertebral body. In some embodiments, the threshold load-bearing amount is selected based at least in part to achieve one or more target outcomes, such as desired fusion or other biomechanics, avoiding subsidence, or the like. In some embodiments, the threshold load-bearing amount of the central region is at least 70%, at least 75%, at least 80%, at least 85%, at least 90%, or at least 95% of the central region, and/or any of the other values identified throughout this Detailed Description. Although described with reference to the superior endplate 410, the inferior endplate 412 can similarly be designed to account for patient anatomy, including the cortical rim and/or a load-bearing threshold of a central region of the corresponding vertebral body endplate.

In some embodiments, the size and/or shape of the superior endplate 410 and/or the inferior endplate 412 can be based on simulations of various outcomes. For example, when designing the implant 400, a user can request, access, or run one or more simulations of virtual candidate implants having different superior endplate 410 and/or inferior endplate 412 sizes, shapes, and/or footprints to determine the likelihood of various post-implant events occurring. Representative events include, but are not limited to, subsidence, expulsion, bony ingrowth, fusion, target biomechanics, or the like. Based on the simulations, a particular one of the candidate implants can be selected (e.g., a candidate implant that meets threshold scores for likelihood of subsidence, likelihood of bony ingrowth, etc.). Alternatively, if none of the candidate implants meet the threshold scores, then the candidate implants can be redesigned and new simulations conducted. In some embodiments, the simulations can be performed using one or more of the data analysis module 116, the treatment planning module 118, the disease progression module 120, and the intervention timing module 121 of the system 100, described with reference to FIG. 1. Accordingly, in some embodiments the simulations are performed at least in part using a trained machine learning model for simulating surgical outcomes using a virtual model of the patient's spine. A user can request, review, and/or interact with the candidate implants and the simulations using the client computing device, 102, the display 122, and/or the surgical plan review program 125 of the system 100. The simulation process can be repeated until a suitable implant design is identified.

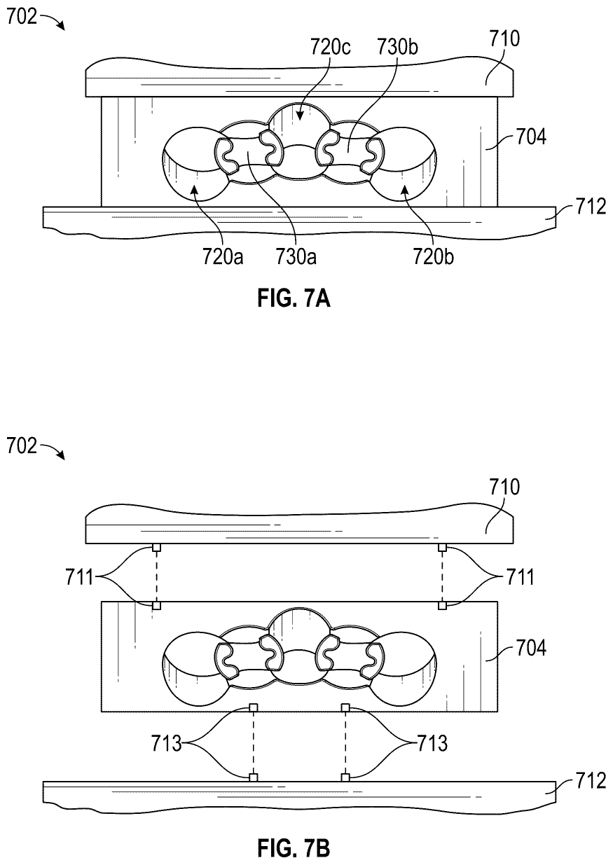

FIGS. 7A and 7B illustrate another patient-specific interbody implant 702 (“the implant 702”) configured in accordance with select embodiments of the present technology. More specifically, FIG. 7A is a partially-schematic front view of the implant 702, and FIG. 7B is a partially exploded view of the implant 702. The implant 702 is a modular implant comprising a body portion 704, a superior endplate 710, and an inferior endplate 712. As shown in FIG. 7A, and similar to the implant 402 of FIG. 4, the body 704 of the implant 702 can include a plurality of lumens (shown as a first lumen 720a, a second lumen 720b, and a third lumen 720c) sized and shaped to receive corresponding fixation elements (e.g., the fixation elements 450 from FIG. 4) to anchor the implant 702 to vertebral structures. The body 704 can also include a first retention mechanism 730a and a second retention mechanism 730b that can be generally similar to or the same as the retention mechanisms 430 described with reference to FIG. 4.

Similar to the implant 402 of FIG. 4, the superior endplate 710 and the inferior endplate 712 can have different dimensions (e.g., different sizes, surface areas, transverse diameters, anteroposterior diameters, etc.). However, unlike the implant 402 in which the superior endplate 410, the inferior endplate 412, and the implant body are a contiguous structure, the body 704, the superior endplate 710, and the inferior endplate 712 of the implant 702 are distinct, modular components. For example, as shown in FIG. 7B, the superior endplate 710 can be coupled to the body 704 via one or more first connection features 711, and the inferior endplate 712 can be coupled to the body 704 via one or more second connection features 713. The first connection features 711 and the second connection features 713 can be formed by any suitable mechanism for securely coupling the endplates to the body 704. Representative examples of connection features include, but are not limited to, threaded connections, magnetic connections, and pin-and-groove, key-and-lock, and other male-female type connections.

In some embodiments, the superior endplate 710 and the inferior endplate 712 can be patient-specific components while the implant body 704 can be a stock (e.g., “off-the-shelf”) component, or selected from a kit having several stock components (e.g., a kit of two, three, four, or more different implant bodies having different heights, depths, etc.). The superior endplate 710 and the inferior endplate 712 can therefore be designed to provide patient-specific topographies and patient-specific footprints as described above with reference to FIGS. 4-6, while the body 704 can be a standardized component that can be manufactured and sterilized in bulk. Without intending to be bound by theory, this may provide certain manufacturing efficiencies, reducing the cost of the implants. In other embodiments, the body 704 can be a patient-specific component, and/or manufactured simultaneously with the superior endplate 710 and the inferior endplate 712 for a particular patient, even if the body 704 does not include any patient-specific features.

The superior endplate 710 and the inferior endplate 712 can be designed with dimensions selected such that, when the implant 702 is implanted between target vertebral bodies, the implant 702 covers a desired percent of the corresponding adjacent vertebral body endplates, and/or sits within a desired distance of a margin of the adjacent vertebral body endplates. For example, the superior endplate 710 can be designed such that, when the implant 702 is implanted, the superior endplate 710 covers at least 80%, at least 85%, at least 90%, at least 91%, at least 92%, at least 93%, at least 94%, or at least 95% of the corresponding vertebral body endplate. As another example, the superior endplate 710 can be designed such that, when the implant 702 is implanted, a posterior edge of the superior endplate 710 is spaced apart from a posterior margin of the corresponding vertebral endplate by about 3 mm or less, by about 2.5 mm or less, by about 2 mm or less, or by about 1.5 mm or less. The inferior endplate 712 can be designed with similar dimensions. The superior endplate 710 and the inferior endplate 712 can also have surfaces with a patient-specific topographies contoured to match specific portions of the surface of the vertebral body endplates.

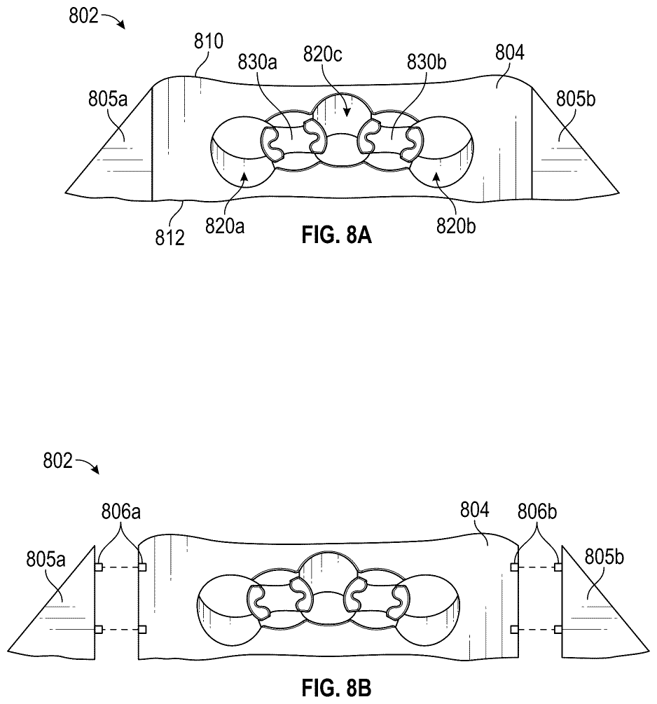

FIGS. 8A and 8B illustrates another patient-specific interbody implant 802 (“the implant 802”) configured in accordance with select embodiments of the present technology. More specifically, FIG. 8A is a partially-schematic front view of the implant 802, and FIG. 8B is a partially exploded view of the implant 802. The implant 802 includes a body portion 804 having a superior endplate 810 and an inferior endplate 812. As shown in FIG. 8A, and similar to the implant 402 of FIG. 4, the body 804 of the implant 802 can include a plurality of lumens (shown as a first lumen 820a, a second lumen 820b, and a third lumen 820c) sized and shaped to receive corresponding fixation elements (e.g., the fixation elements 450 from FIG. 4) to anchor the implant 802 to vertebral structures. The body 804 can also include a first retention mechanism 830a and a second retention mechanism 830b that can be generally similar to or the same as the retention mechanisms 430 described with reference to FIG. 4.

Similar to the implant 402 of FIG. 4 and the implant 702 of FIGS. 7A and 7B, the superior endplate 710 and the inferior endplate 712 can have different dimensions (e.g., different sizes, surface areas, transverse diameters, anteroposterior diameters, etc.). However, unlike the implant 402 of FIG. 4 and the implant 702 of FIGS. 7A and 7B, the implant 802 achieves this via a first lateral extender 805a and a second lateral extender 805b (collectively referred to as “the lateral extenders 805”). When coupled to the body 804, the lateral extenders 805a can increase one or more dimensions of the inferior endplate 812 (e.g., increase a transverse diameter of the inferior endplate 812) without changing, or without significantly changing, the corresponding dimension of the superior endplate 810. Accordingly, the lateral extenders 805 have a generally triangular cross-sectional shape, increasing in width in the superior to inferior direction. The size and shape of the lateral extenders 805 can be designed based on the desired footprint of the inferior endplate 812. Although shown as increasing the footprint of the inferior endplate 812, in some embodiments, the lateral extenders 805 can also be used to increase the footprint of the superior endplate 810, in addition to or in lieu of increasing the footprint of the inferior endplate 812.

The lateral extenders 805 can be designed with dimensions selected such that, when the implant 802 is implanted between target vertebral bodies, the implant 802 covers a desired percent of the adjacent vertebral endplates, and/or sits within a desired distance of a margin of the adjacent vertebral endplates. For example, the lateral extenders 805 can be designed such that, when the implant 802 is implanted, the inferior endplate 812 covers at least 80%, at least 85%, at least 90%, at least 91%, at least 92%, at least 93%, at least 94%, or at least 95% of the corresponding vertebral body endplate. As another example, the lateral extenders 805 can be designed such that, when the implant 802 is implanted, a lateral edge of the implant 802 is spaced apart from a lateral margin of the corresponding vertebral endplate by about 3 mm or less, by about 2.5 mm or less, by about 2 mm or less, or by about 1.5 mm or less. The lateral extenders 805 can also have surfaces with a patient-specific topography contoured to match specific portions of the surface of the vertebral body endplate.

The present technology also provides implants designed for corpectomy procedures. A corpectomy is a surgical procedure in which a substantial portion of the anterior side of a vertebral body is removed from the patient, and is often performed to relieve pressure on the spinal cord and associated nerves. Once the vertebral body is removed, a fusion device can be inserted into the spinal column to provide structural support and provide a cavity for receiving bone graft material to promote fusion. However, unlike standard spinal fusion devices, fusions devices designed for use in a corpectomy procedure generally have a greater height because they much span multiple vertebral levels. For example, if a corpectomy procedure removed the anterior portion of the C3 vertebral body to relieve pressure on a patient's cervical spinal cord, an interbody fusion device must be designed to span from the inferior side of the C2 vertebral body to the superior surface of the C4 vertebral body. Any of the implants described herein, including those described above with reference to FIGS. 4-8B, can be designed as corpectomy devices.

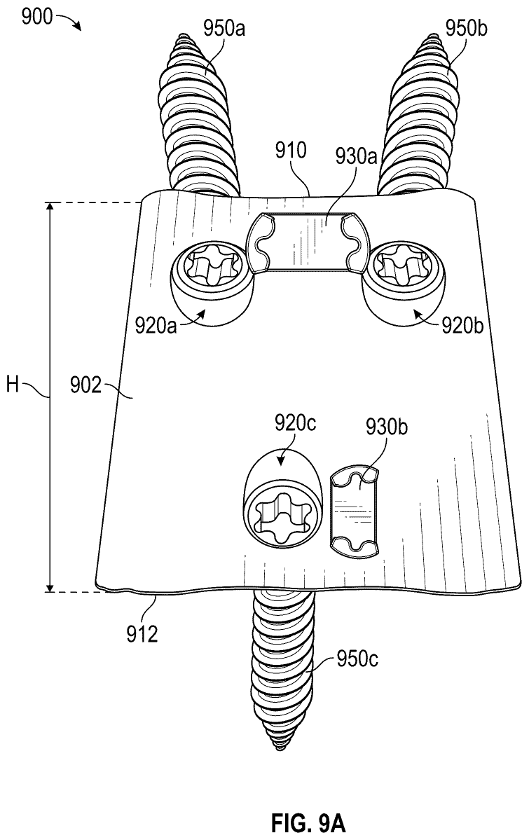

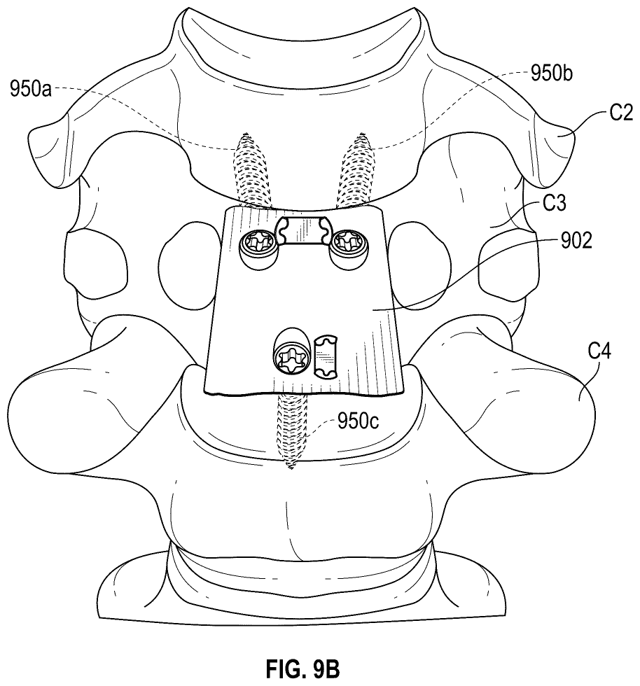

FIG. 9A is a front view of a patient-specific spinal fusion device 900 (“the device 900”) designed for a corpectomy spinal fusion procedure and configured in accordance with select embodiments of the present technology. Similar to the device 400 of FIG. 4, the device 900 can include a patient-specific interbody implant 902 and a plurality of fixation elements 950 (show individually as a first fixation element 950a, a second fixation element 950b, and a third fixation element 950c). Also similar to the implant 402 of FIG. 4, the implant 402 can include a plurality of lumens (shown as a first lumen 920a, a second lumen 920b, and third lumen 920c) sized and shaped to receive the corresponding fixation elements to anchor the implant 902 to vertebral structures. The implant 902 can also include a first retention mechanism 930a for releasably locking the first fixation element 950a in the first lumen 920a and the second fixation element 950b in the second lumen 920b, and a second retention mechanism 930b for releasably locking the third fixation element 950c in the third lumen 920c (collectively referred to as “the retention mechanisms 930”). Similar to the retention mechanisms described with reference to FIG. 4, the retention mechanisms 930 can each include a rotatable cam, head, tab, or other structure that can be rotated between a first, unlocked position that does not interfere with the corresponding lumen(s), and a second, locked position that at least partially blocks an opening to the corresponding lumen(s) 920.

The implant 902 further includes a superior surface or endplate 910 and an inferior surface or endplate 912. Similar to the devices described throughout this Detailed Description, the superior endplate 910 can have a different size/surface area (e.g., by virtue of having different transverse and/or anteroposterior diameters) than the inferior endplate 912. Both the superior endplate 910 and the inferior endplate 912 can also be designed to have a patient-specific topography that is contoured to match (e.g., mate with) the corresponding vertebral target structures. However, the distance between the superior endplate 910 and the inferior endplate 912 (e.g., the height H of the implant) is greater than a typical interbody device. The height of the implant 902 can be patient-specific based on (a) the spacing between vertebral bodies at a target implant location, and (b) any desired anatomical correction. For example, in the context of a cervical corpectomy procedure, the implant 902 may have a height H of between about 10 mm and about 20 mm, or between about 12 mm and about 18 mm, or about 13 mm, about 14 mm, about 15 mm, about 16 mm, or about 17 mm.

FIG. 9B illustrates the device 900 implanted in a patient's spine. In the illustrated embodiment, an anterior portion of the C3 vertebral body has been removed before the device 900 was implanted, and the device 900 extends between the inferior endplate of the C2 vertebral body and the superior endplate of the C4 vertebral body. Accordingly, in the illustrated embodiment, the superior endplate 910 can be designed based on the desired coverage and topography of the inferior endplate of the C2 vertebral body, and the inferior endplate 912 can be designed based on the desired coverage and topography of the superior endplate of the C4 vertebral body. The height H can be designed based on a desired post-surgical spacing between the C2 and C4 vertebral bodies. Although shown as being implanted between the C2 and C4 vertebral bodies, the device 900 can be implanted at other locations along the patient's spinal column. In some embodiments, the device 900 can be designed to extend across two or more vertebral bodies, e.g., for corpectomy procedures that involve the removal of multiple vertebral bodies.

Any of the devices and implants described herein can also be implanted with one or more additional implants or components. For example, in some embodiments multiple interbody devices (e.g., multiple devices 400 of FIG. 4) can be implanted at different levels of the patient's spinal column, such as at two or more of C1-C2, C2-C3, C3-C4, C4-C5, etc. In such embodiments, each interbody device can be specifically designed for the vertebral level it will be implanted at. Thus, each interbody device will generally have different dimensions, even though they are intended to be implanted in the same patient.

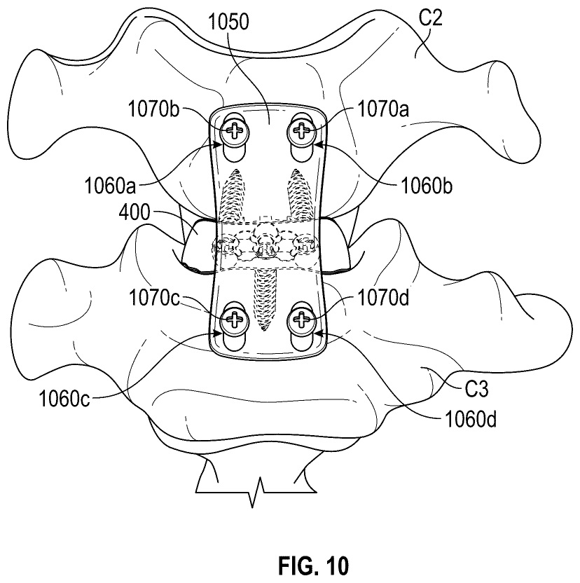

In some embodiments, the interbody devices described herein can be implanted in combination with an anterior plate. For example, FIG. 10 illustrates the device 400 of FIG. 4 implanted between the C2 and C3 vertebral bodies in combination with a plate 1050. The plate 1050 can be sized and shaped to extend across the intervertebral space in which the device 400 is implanted (e.g., the C2-C3 intervertebral space), between a general midpoint of the C2 vertebral body and a general midpoint of the C3 vertebral body. The plate 1050 can further include a plurality of slots 1060a-d sized and shaped to receive a plurality of fixation elements 1070a-d for securing the plate to the adjacent vertebral bodies. For example, in the illustrated embodiment the first slot 1060a and the second slot 1060b are positioned proximate the C2 vertebral body such that the first fixation element 1070a and the second fixation element 1070b inserted through the first slot 1060a and the second slot 1060b, respectively, anchor the plate 1050 to the C2 vertebral body. The third slot 1060c and the fourth slot 1060d are positioned proximate the C3 vertebral body such that the third fixation element 1070c and the fourth fixation element 1070d inserted through the third slot 1060c and the fourth slot 1060d, respectively, anchor the plate 1050 to the C3 vertebral body. The plate 1050 can promote stability of the spinal column and/or reduce the likelihood of expulsion of the interbody device 400.

In some embodiments, the plate 1050 can be patient-specific. For example, the plate 1050 can have one or more surfaces (e.g., posterior facing surfaces, not visible in FIG. 10) that have a patient-specific topography contoured to match (e.g., mate with) a topography of the vertebral bodies the surfaces contact. The plate 1050 can also have patient specific dimensions to ensure that the slots 1060a-d align with dense portions of the corresponding vertebral bodies to ensure the fixation elements 1070a-d are inserted into a strong portion of the vertebral bodies. Additional features of patient-specific plates that can be used in combination with the present technology are described in U.S. Patent Application Publication Nos. 2022/0160518 and 2022/0401150, the disclosure of which are incorporated by reference herein in their entireties.

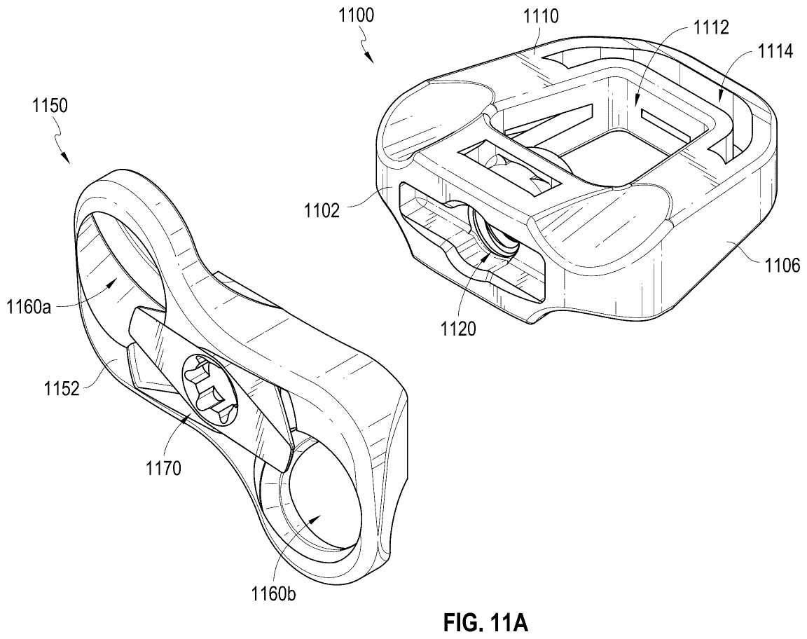

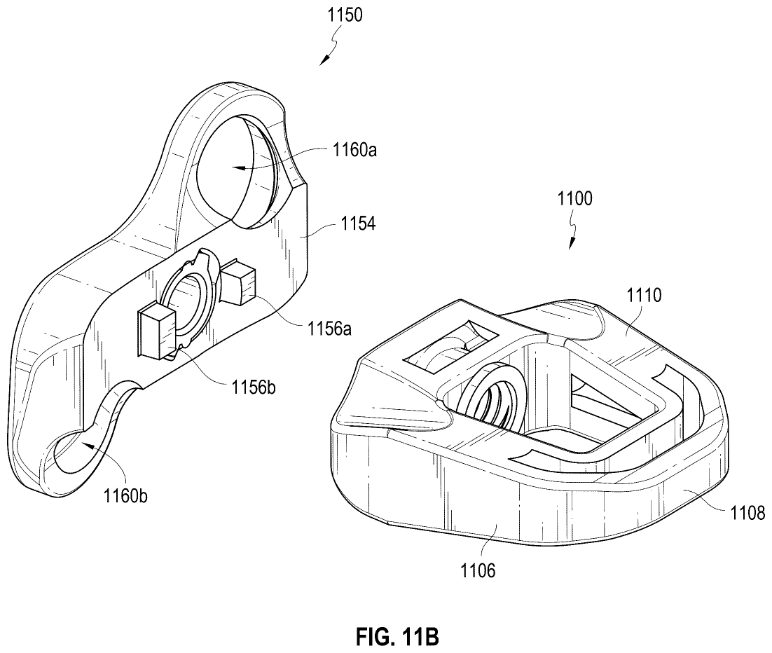

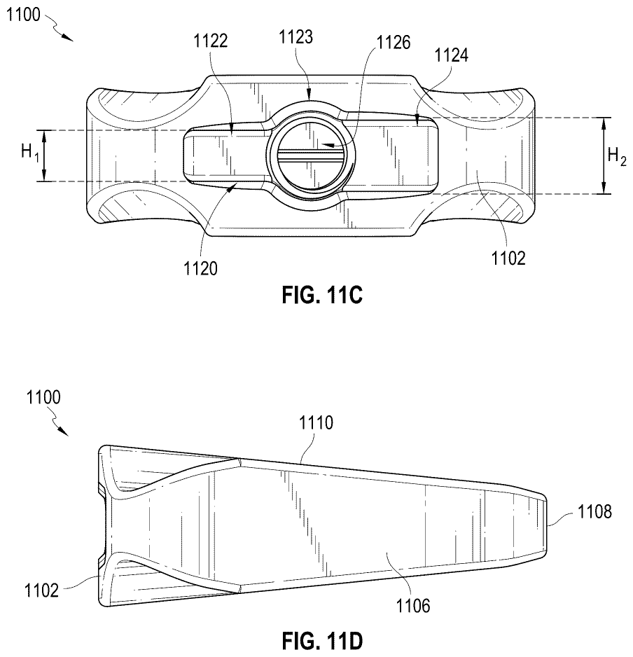

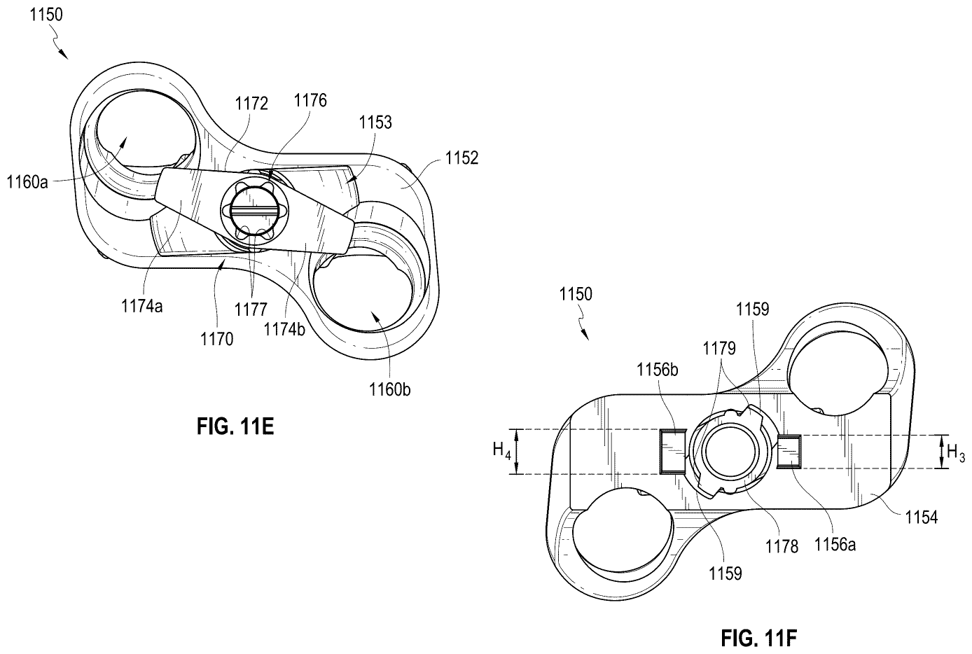

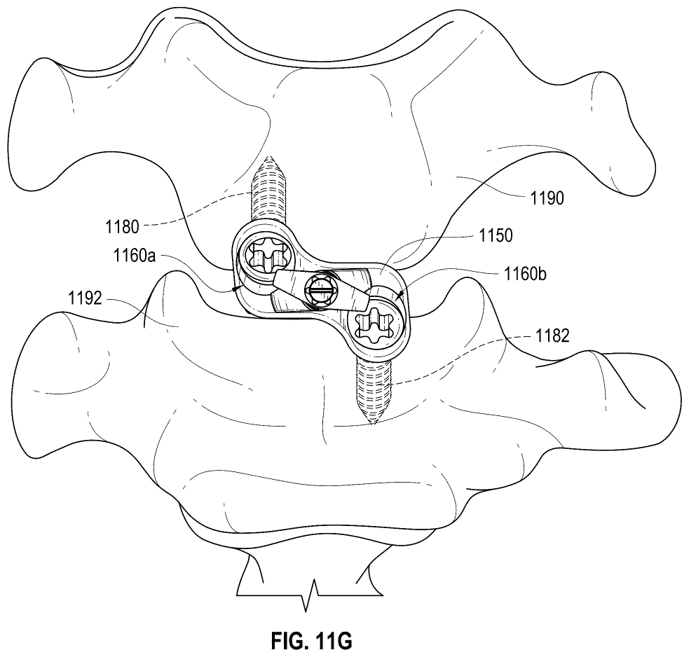

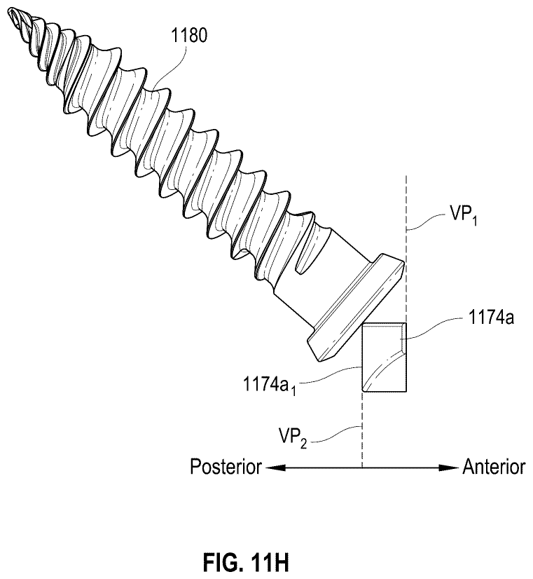

FIGS. 11A-11H illustrate a patient-specific interbody device or cage 1100 (“the cage 1100”) and a patient-specific anterior plate 1150 (“the plate 1150”) configured in accordance with embodiments of the present technology. More specifically, FIG. 11A is a perspective view of the cage 1100 and the plate 1150 in an uncoupled configuration, and FIG. 11B is another perspective view of the cage 1100 and the plate 1150 in the uncoupled configuration, but rotated 180 degrees relative to the view of FIG. 11A. FIGS. 11C and 11D are front and side views, respectively, of the cage 1100, and FIGS. 11E and 11F are front and rear views, respectively, of the plate 1150. FIG. 11G illustrates the cage 1100 and the plate 1150 implanted along a portion of a patient's spine. FIG. 11H is a side view of a portion of the plate 1150 taken along the direction indicated in FIG. 11G.

Referring collectively to FIGS. 11A and 11B, the cage 1100 includes a superior surface 1110, an inferior surface (not visible in FIG. 11A or 11B), an anterior surface 1102, a posterior surface 1108, and two lateral surfaces 1106. The cage 1100 can have a central opening 1112 extending through both the superior surface 1110 and the inferior surface to promote vertebral fusion. The cage 1100 may also include one or more perimeter openings or recesses 1114 extending partially or fully between the superior surface 1110 and the inferior surface. The central opening 1112 and/or the perimeter openings 1114 can optionally receive a graft material when implanted in the patient to promote vertebral fusion. The cage 1100 can include other features for promoting fusion and/or improving the biomechanical properties of the cage 1100, such as one or more lattice structures. As described throughout this Detailed Description, the superior surface 1110 and the inferior surface can each have a patient-specific topography designed to match a corresponding topography of the vertebral body endplate that it is designed to contact. The overall footprint, size, and shape of the cage 1100 can also be patient-specific, as described with respect to the interbody devices described with respect to FIGS. 4-10. For example, as shown in FIG. 11D, the cage 1100 can be tapered between the anterior surface 1102 and the posterior surface 1108, such that the height of the cage 1100 is greater in the anterior region than the posterior region. The amount and rate of tapering can be tailored to individual patients in accordance with a planned surgical correction, and can be determined using the techniques described above with reference to FIGS. 1-3.

Unlike the other interbody implants described previously, the cage 1100 does not include any holes for receiving fixation elements (e.g., screws), and thus does not include a retention mechanism for reducing the likelihood of back-out of the fixation element. Instead, the anterior surface 1102 includes a recess or other cavity 1120. As best shown in FIG. 11C, the recess 1120 can be an inset of the anterior face 1102 of the cage 1100. The recess 1120 can extending in a generally horizontal manner (e.g., parallel to the superior surface 1110 and the inferior surface of the cage 1100) and include a first region 1122 (e.g., a first lateral wing), a second region 1124 (e.g., a second lateral wing) opposite the first region 1122, and a central region 1123 between the first region 1122 and the second region 1124. The first region 1122 and the second region 1124 can have different properties. For example, in the illustrated embodiment the first region 1122 has a first height H1 and the second region 1124 has a second height H2 that is different than the first height H1. The second height H1 may be between about 20% and about 100% larger than the first height H1. In addition to or in lieu of having different heights, the first region 1122 and the second region 1124 can have different widths, depths, shapes, or other features. As described below, the different characteristics of the first region 1122 and the second region 1124 can help ensure proper alignment between the cage 1100 and the plate 1150, when implanted. The central region 1123 can be round and generally align with a receiving aperture 1126 configured to be releasably coupled to an inserter instrument (not shown) for implanting the cage 1100. The receiving aperture 1126 can therefore include threading or other element for releasably coupling to the inserter instrument.

Referring again to FIGS. 11A and 11B, the plate 1150 includes an anterior surface 1152 (FIG. 11A) designed to face away from the cage 1100 and a posterior surface 1154 (FIG. 11B) designed to face toward the cage 1100, when the plate 1150 and the cage 1100 are oriented in the configuration they are intended to be implanted in. A first opening 1160a and a second opening 1160b (collectively referred to as the “openings 1160”) extend between the anterior surface 1152 and the posterior surface 1154. As shown in FIG. 11G, the openings 1160 can be sized and shaped to receive corresponding fixation elements 1180, 1182 (e.g., a screw) to fix the plate 1150 to a superior vertebral body 1190 and an inferior vertebral body 1192. The openings 1160 can be at least partially vertically offset from a horizontal axis extending through a midline of the plate 1152. For example, the first opening 1160a can be offset superiorly relative to the horizontal midline axis, and the second opening 1160b can be offset inferiorly relative to the horizontal midline axis. In the illustrated embodiment, the openings 1160 are offset by the same distance relative to the horizontal midline axis, although in other embodiments the openings 1160 can be offset by different distances based on variable patient-specific anatomy (e.g., to ensure that the openings 1160 align with a portion of the vertebral body that is suited to receive a fixation element). For example, one of the openings 1160 may be offset by between 1 mm and 20 mm more than the other of the openings 1160. Accordingly, the plate 1150 can also be patient-specific.

The plate 1152 further includes a retention mechanism 1170 for retaining the fixation elements (not shown) in the openings 1160. Similar to the retention mechanism 430 described with reference to FIG. 4, the retention mechanism can be selectively rotated or otherwise manipulated between a first, unlocked configuration (not shown) that permits a user to insert the fixation elements 1180, 1182 (FIG. 11G) into the openings 1160, and a second, locked configuration (shown in FIG. 11A) that locks the fixation elements 1180, 1182 in the openings 1160. For example, as best shown in FIG. 11E, the retention mechanism 1170 can include a rotating cam 1172 having a first extension 1174a and a second extension 1174b (referred to collectively as “the extensions 1174”). When in the locked configuration, the first extension 1174a can at least partially cover the first opening 1160a and the second extension 1174b can at least partially cover the second opening 1160b. When rotated to the unlocked configuration, the extensions 11674 can be positioned within a recess 1153 on the anterior surface 1152 of the plate 1150, leaving the openings 1160 unblocked. In some embodiments, an anterior surface of the cam 1172 is flush with (e.g., in plane with) the anterior surface 1152 of the plate 1150 (e.g., the cam 1172 does not extend anteriorly relative to the plate 1150). This may reduce the likelihood of the cam 1172 irritating adjacent patient anatomy, such as the esophagus or trachea.

Because the cam 1172 sits within the recess 1153, and because the fixation elements 1180, 1182 (FIG. 11G) are angled relative to the plate 1150, the cam 1172 may not extend over an entirety of a head of the fixation elements. Indeed, as shown in FIG. 11H, a posterior surface 1174a₁ of the first extension 1174 can extend within a first generally vertical plane VP₁ that is posterior to a second generally vertical plane VP₂ extending tangentially relative to an anterior-most aspect of the fixation element 1180. This allows tissue (e.g., tissue of the esophagus) to atraumatically slide along components of the implant. The first extension 1174 can sufficiently overlap the fixation element 1180 to reduce the likelihood of back-out of the fixation element 1180. Moreover, both the first vertical plane VP₁ and the second vertical plane VP₂ can be posterior to (or co-planar with) the anterior surface 1152 of the plate 1150.

Returning to FIGS. 11E and 11F, the rotating cam 1172 can further include a central opening 1176 with a plurality of drive features 1177 (e.g., alternating protrusions and recesses) that enable a tool (not shown) to rotate the cam 1172 between the locked configuration and the unlocked configuration. The central opening 1176 can also be sized and shaped to receive an inserter instrument for delivering the plate 1150 to a target implant location. In some embodiments, the inserter instrument can be the same as the tool used to rotate the cam 1172 between the unlocked and locked configurations. Further yet, in some embodiments the central opening 1176 can be aligned with (e.g., share a common central axis with) the aperture 1126 on the cage 1100, such that a single inserter instrument can simultaneously extend through the opening 1176 and the aperture 1126, and thus be simultaneously coupled to plate 1150 and cage 1100 (e.g., to enable the plate 1150 and the cage 1100 to be implanted at the same time).

The retention mechanism 1170 further includes a shaft 1178 extending posteriorly from the cam 1172. The shaft 1178 can include a ridge or other feature 1179 that sits within a corresponding groove 1159 in the plate to define a range of motion for rotating the cam 1172. Additional details of retention mechanisms that can be used with the plate 1150 are described in International Patent Application Publication No. WO2024/148108, the disclosure of which was previously incorporated by reference herein in its entirety.

As best shown in FIGS. 11B and 11F, the plate 1150 includes a first coupling feature 1156a and a second coupling feature 1156b (collectively referred to as “the coupling features 1156”) extending posteriorly from the posterior surface 1154 as tabs, projections, bumps, etc. The coupling features 1156 can have various three-dimensional shapes, including cuboid, pyramidal, spherical, irregular, or the like. Notably, the coupling features 1156 have different shapes and/or sizes. For example, in the illustrated embodiment the first coupling feature 1156a has a first height H3 and the second coupling feature 1156b has a second height H4 that is different than the first height H3. The second height H4 may be between about 20% and 100% greater than the first height H3. In addition to or in lieu of having different heights, the coupling features 1156 can have different widths, depths, shapes, or other features. As described in detail below, providing the coupling features 1156 with different shapes/sizes enables a keyed fit between the plate 1150 and the cage 1100 to ensure the plate 1150 and cage 1100 are implanted at the intended relative orientation.

The shape and size of the coupling features 1156 correspond to the shape and size of the first region 1122 and the second region 1124 of the recess 1120 of the cage 1100 (FIG. 11C). That is, the first height H3 of the first coupling feature 1156a can correspond to (e.g., be equal or slightly less than) the first height H1 of the first region 1122, and the second height H4 of the second coupling feature 1156b can correspond to (e.g., be equal or slightly less than) the second height H2 of the second region 1124. In this manner, the first coupling feature 1156a can be inserted into the first region 1122 of the recess 1120, and the second coupling feature 1156b can be inserted into the second region 1124 of the recess 1120. In other embodiments, the coupling features can have different, but complementary shapes (e.g., the first coupling feature 1156a and the first region 1122 are pyramidal, and the second coupling feature 1156b and the second region 1124 are cuboid). The complementary fit between the coupling features 1156 on the plate 1150 and the recess 1120 on the cage 1100 are designed to ensure that the cage 1100 and the plate 1150 are implanted at the proper orientation relative to each other.

When the cage 1100 and the plate 1150 are implanted along a patient's spine as shown in FIG. 11G, the coupling features 1156 are configured to sit at least partially within the recess 1120. This can help ensure the cage 1100 and the plate 1150 retain a proper orientation relative to one another, and further reduce the likelihood of the cage 1100 being expelled from the intervertebral disc space. However, the plate 1150 need not be fixedly coupled to the implant 1100 via the coupling features 1156. The coupling features 1156 instead sit within, but are not otherwise fixedly secured to, the recess 1120. This can enable micromovements between the plate 1150 and the cage 1100, e.g., to accommodate slight positional changes in response to bony fusion and/or patient movement.

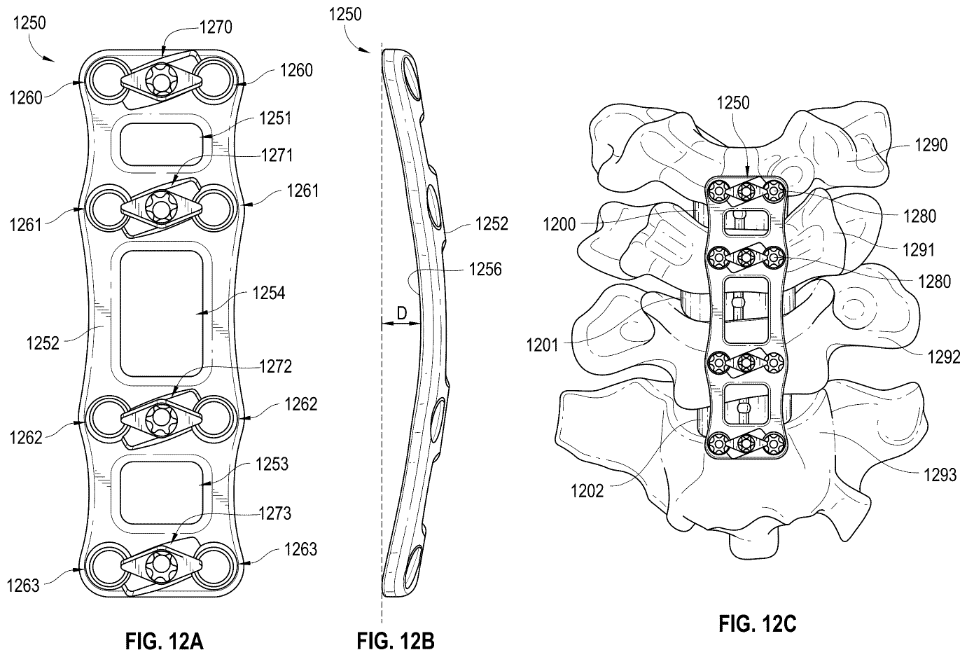

FIGS. 12A and 12B illustrate a front and side view, respectively, of another patient-specific plate 1250 (“the plate 1250”) configured in accordance with embodiments of the present technology. FIG. 12C illustrates the plate 1250 implanted along a vertebral column of a patient. Referring first to FIG. 12A, the plate 1250 can be sized and shaped to extend multiple vertebral levels when implanted in a patient, and can be configured to be affixed to a plurality of vertebral bodies. For example, the plate can include a first pair of openings 1260 and a corresponding first retention mechanism 1270, a second pair of openings 1261 and a corresponding second retention mechanism 1271, a third pair of openings 1262 and a corresponding third retention mechanism 1272, and a fourth pair of openings 1263 and a corresponding fourth retention mechanism 1273. As shown in FIG. 12C, each pair of openings 1260-1263 can be aligned with a different vertebral body 1290-1293 when implanted in a patient. The openings 1260-1263 can be sized and shaped to receive fixation elements 1280 therethrough for fixedly coupling the plate to the vertebral body that the openings 1260-1263 are aligned with. Although shown as being coupled to four different vertebral bodies 1290-1293, in other embodiments the plate 1250 can be configured to be coupled to more or fewer vertebral bodies, such as two, three, five, six, or more.

As shown in FIG. 12A, the plate 1250 can further include openings or windows extending between an anterior surface 152 and a posterior surface (not visible in FIG. 12A) of the plate 1250. For example, the plate 1250 includes a first window 1251 positioned between the first pair of openings 1260 and the second pair of openings 1261, a second window 1254 positioned between the second pair of openings 1261 and the third pair of openings 1262, and a third window 1253 positioned between the third pair of openings 1262 and the fourth pair of openings 1263. As shown in FIG. 12C, the windows are sized and shaped to enable a user to visualize an intervertebral space between adjacent vertebral bodies 1290-1293 when the plate 1250 is implanted. Thus, each of the windows can have a different size and/or shape based on patient-specific anatomy to ensure that the windows align with the intended intervertebral space when the plate 1250 is implanted.

As shown in FIG. 12B, the plate 1250 can have a slight arcuate or curved shape, such that a posterior surface 1256 of the plate is at least partially concave. In some embodiments, the plate 1250 has a concavity with a maximum dimension D (e.g., depth) of between about 5 mm and 50 mm, or between about 5 mm and about 25, or between about 5 mm and about 15 mm. The curvature can be a patient-specific curvature based on an expected curvature of the patient's spinal column when positioned in the target surgical correction (FIGS. 1-3). Accordingly, an upper or lower portion of the plate 1250 may be curved different amounts to correspond to the expected curvature.

The plate 1250 can be used with any of the intervertebral implants and cages described in this Detailed Description. For example, FIG. 12C illustrates the plate 1250 implanted in combination with a first patient-specific cage 1200 between a first vertebral body 1290 and a second vertebral body 1291, a second patient-specific cage 1201 between the second vertebral body 1291 and a third vertebral body 1292, and a third patient-specific cage 1203 between the third vertebral body 1292 and a fourth vertebral body 1293. Each of the cages 1200-1202 can be patient-specific, and thus each of the cages 1200-1202 can have different sizes and shapes. For example, each cage 1200-1202 may have a different superior and inferior endplate footprint designed to match the corresponding vertebral body endplate that it will contact when implanted, as described in detail with reference to FIGS. 4-10. That is, the first cage 1200 can include a first superior endplate and a first inferior endplate having different sizes/footprints to cover a target amount of the vertebral body endplate it is designed to contact, and/or to sit within a target distance of a margin of the vertebral body endplate it is designed to contact. Similarly, the second cage 1201 can include a second superior endplate and a second inferior endplate having different sizes/footprints, and the third cage 1202 can have a third superior endplate and a third inferior endplate having different sizes/footprints. Moreover, the footprints for each of the first, second, and third superior and inferior endplates may all be different from each other such that there are six unique endplate footprints for the three cages 1200-1203, consistent with the implants described with reference to FIGS. 4-10. Accordingly, the present technology can includes kits of patient-specific implants in which each implant includes not only different superior and inferior endplates, but also that includes different endplates than other implants within the kit.

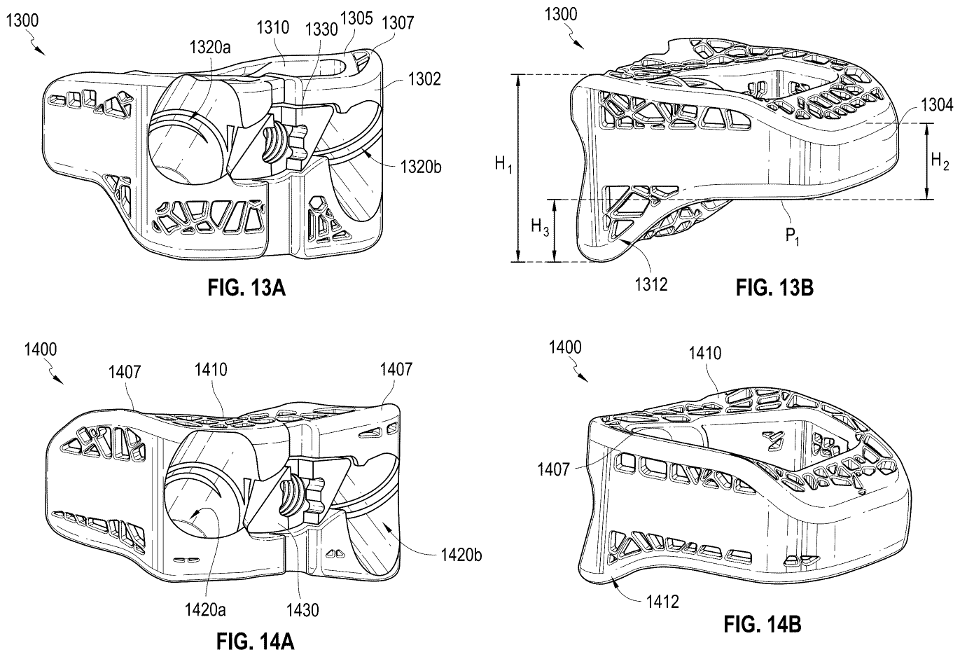

FIGS. 13A and 13B illustrate another patient-specific intervertebral implant or cage 1300 (“the implant 1300”) configured in accordance with embodiments of the present technology. The implant 1300 can include certain features generally similar to the implant 400 described with reference to FIG. 4. For example, the implant 1300 can include a first opening or lumen 1320a and a second opening or lumen 1320b sized and shaped to receive corresponding fixation elements (e.g., screws) to secure the implant 1300 to adjacent vertebral bodies. The implant 1300 can further include a retention mechanism 1330a to hold fixation elements within the first lumen 1320a and the second lumen 1320b. The retention mechanism 1330a can be generally similar to or the same as the retention mechanisms described throughout this Detailed Description, and/or generally similar to or the same as the retention mechanisms described in International Patent Application Publication No. WO2024/148108, previously incorporated by reference herein.

The implant 1300 can have perimeter features for improved endplate loading and seating. For example, as shown in FIG. 13A, one or more edges of the implant 1300 can include a load-bearing or seating portion 1305 having a semi-curved perimeter ridge or rim 1307 extending upwardly therefrom (which can collectively be referred to as a “cortical rim receiving portion”). The perimeter ridge 1307 may extend upwardly between about 1 mm and about 8 mm, or between about 1 mm and about 5 mm, or between about 1 mm and about 3 mm, relative to the load-bearing portion 1305. In some embodiments, the load-bearing portion 1305 can be configured to provide load-bearing support to the corresponding vertebral body (e.g., along the patient's cortical rim), and the semi-curved perimeter ridge 1307 can trace vertebral anatomy outwardly from the load-bearing regions to reduce the likelihood of the implant 1300 shifting relative to the vertebral body. In other embodiments, the perimeter ridge 1307 can itself function as a load-bearing portion of the implant 1300. For example, the load can be disturbed across both the load-bearing portion 1305 and the perimeter ridge 1307 (e.g., distributed across the entire cortical rim receiving portion such that no single portion of the implant receives a load greater than a preset threshold). The perimeter ridge 1307 can extend around at least 25%, at least 50%, at least 75%, at least 85%, or at least 90% of the superior surface 1310 of the implant 1300.

The implant 1300 can also be designed to provide improved endplate coverage and seating. For example, as best shown in FIG. 13B, the anterior surface 1302 of the implant 1300 can have a first height H₁, and the posterior surface 1304 of the implant 1300 can have a second height H₁. Based on patient-anatomy and the target anatomical correction (FIGS. 1-3), the first height H₁ can be between 50% to 200% greater than the first height H₂. However, instead of having a steady taper between the anterior surface 1302 and the posterior surface 1304, the implant 1300 includes a wing-like extension 1312 extending from an inferior portion of the implant 1300. That is, the inferior surface of the endplate can be generally planar along a plane P₁ at a posterior portion of the implant 1300, and the wing-like extension 1312 can extend inferiorly from the plane P₁. The extension 1312 can have a third height H₃ at the anterior surface 1302. In seme embodiments, the third height H₃, which contributes to the overall first height H₁ of the anterior surface 1302, can be equal to or greater than the second height H₂ of the posterior surface 1304. In other embodiments, the third height H₃ is less than the second height H₂. In some embodiments, a ratio of the third height H₃ to the second height H₂ is between about 0.5:1 and 1.5:1, or between about 0.8:1 and 1.2:1, or about 1:1. Because the implant 1300 can be patient-specific, the exaction ratio can be dictated by the patient's anatomy following the planned surgical correction.

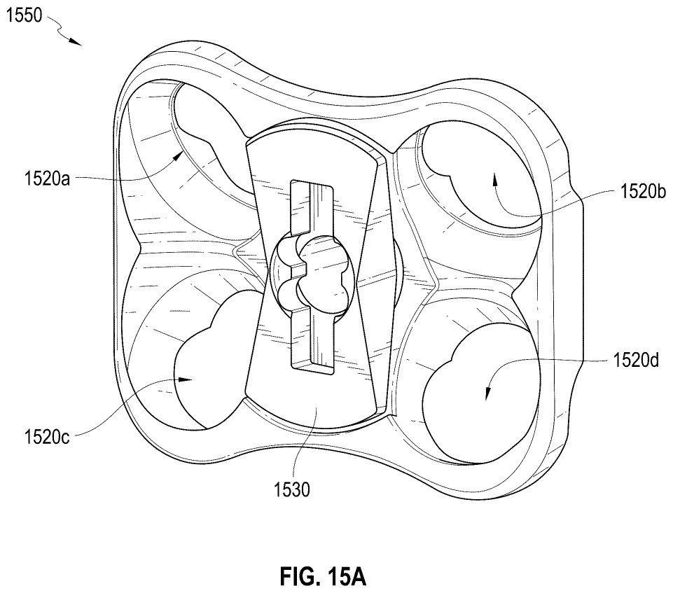

FIGS. 14A and 14B illustrate yet another patient-specific intervertebral implant or cage 1400 (“the implant 1400”) configured in accordance select embodiments of the present technology. The implant 1400 can be generally similar to the implant 1300 of FIGS. 13A and 13B, and can include a first lumen 1420a for receiving a first fixation element, a second lumen 1420b for receiving a second fixation element, and a retention mechanism 1430. At least the superior surface 1410 of the implant 1400 can include a semi-curved perimeter ridge or rim 1407, similar to the perimeter ridge 1307 of the implant 1300. As best shown in FIG. 14B, the superior surface 1410 of the implant 1400 can be concave, such that the perimeter rim 1407 extends superiorly to the rest of the implant 1300. A inferior surface of the implant 1400 may have a similar concavity and perimeter rim. This may (a) improve load-bearing characteristics of the implant 1300 by shifting loads to or adjacent to the perimeter rim 1407, thus seating load-bearing regions along specific regions of the vertebral bodies (e.g., the cortical rim), and/or (b) enable increased endplate coverage by increasing the overall footprint of the implant 1300 while retaining the patient-specific topography of implant endplate surfaces. The perimeter rims 1407 can be configured to contact most of or substantially all of a length of the cortical rim to hold the vertebral body at a target position during implantation.

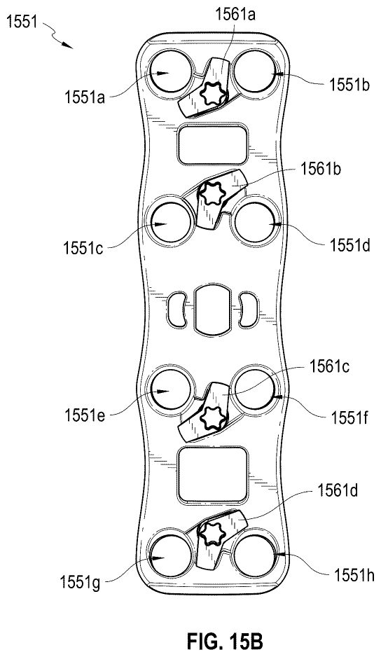

As set forth throughout this Detailed Description, any of the interbody implants described herein can be used in combination with an anterior plate. FIGS. 15A and 15B illustrate additional embodiments of patient-specific anterior plates configured in accordance with embodiments of the present technology. FIG. 15A illustrates a single-level patient-specific plate 1550. Similar to the plate 1050 of FIG. 10, the plate 1550 of FIG. 15A includes four lumens or openings 1560a-d for receiving corresponding fixation elements. The plate 1050 further includes a retention mechanism 1570, which can be generally similar to or the same as any of the retention mechanisms described herein. FIG. 15B illustrates a multi-level patient-specific plate 1551 that is sized to extend across three vertebral segments, and accordingly has eight lumens or openings 1561a-h and four retention mechanisms 1571a-d. As one skilled in the art will appreciate, the present technology can include plates extending across any number of vertebral segments, such as one, two, three, four, five, six, or more. The particular number of vertebral segments can depend on the patient-specific surgical plan (FIGS. 1-3).

As one skilled in the art will appreciate, the patient specific spinal fusion devices described with respect to FIGS. 4-15B are provided as representative embodiments. The present technology includes variations of these embodiments, including devices with combinations of features described with respect to FIGS. 4-15B and devices with only some of the features described with respect to FIGS. 4-15B.

### D. Select Methods of Designing Patient-Specific Implants with Different Endplate Dimensions and Features

As set forth in Section C of this Detailed Description, the present technology includes interbody implants with patient-specific endplates that include both (a) patient-specific dimensions to provide for a desired coverage of the corresponding vertebral body endplate, and (b) patient-specific topography to provide a good fit with the corresponding vertebral body endplate. The present technology further includes methods of designing such implants.

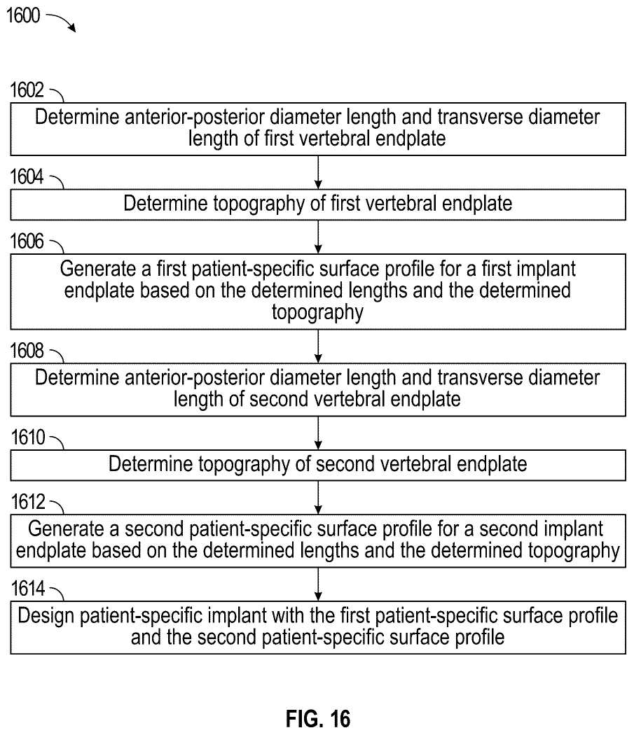

For example, FIG. 16 is a flowchart of a method 1600 of designing a patient-specific interbody implant having a first patient-specific endplate and a second patient-specific endplate in accordance with select embodiments of the present technology. In some embodiments, the operations of method 1600 can be performed as a sub-method of the operation at block 314 of the method 300 described with reference to FIG. 3. That is, each of the operations described below can be performed as part of designing the patient-specific cervical interbody implant based on a selected surgical plan.

The method 1600 can begin at block 1602 by determining an anterior-posterior diameter length and a transverse diameter length of a first vertebral body endplate. In some embodiments, the foregoing lengths can be determined using image data of the patient's spinal anatomy, such as X-ray images, CT images, MRI images, or the like. In other embodiments, the lengths can be determined using a virtual model of the patient's spinal anatomy generated using source X-ray images, CT images, MRI images, or the like. Regardless, in some embodiments, determining an anterior-posterior diameter length and a transverse diameter length includes accessing a segmented image or model of the first vertebral body showing the first vertebral body endplate and adding perimeter profiles to the image or model of the first vertebral body endplate to define the boundaries of the anatomical implant interface.

The method 1600 can continue at block 1604 by determining a topography of the first vertebral endplate. In some embodiments, the foregoing lengths can be determined using image data of the patient's spinal anatomy, such as X-ray images, CT images, MRI images, or the like. In other embodiments, the lengths can be determined using a virtual model of the patient's spinal anatomy generated using source X-ray images, CT images, MRI images, or the like. Regardless, in some embodiments, determining the topography includes using a bounding box centroid to create profile points around the bounding box used to obtain the diameter lengths, and generating an endplate profile.

The method 1600 can continue at block 1606 be generating a first patient-specific profile for the first patient-specific endplate using the lengths determined at bock 1602 and the topography determined at block 1604. For example, the patient-specific profile can include a footprint designed to cover at least 80%, at least 85%, at least 90%, at least 91%, at least 92%, at least 93%, at least 94%, or at least 95% of the endplate. The patient-specific profile can also include a footprint having a transverse diameter length that is between 2-6 mm less than the transverse diameter length of the first vertebral endplate, such that lateral edges of the implant sit between 1-3 mm from the lateral margin of the first vertebral endplate. Similarly, the patient-specific profile can have an anteroposterior diameter length that is between 2-6 mm less than the anterior-posterior diameter length of the first vertebral endplate, such that the anterior and posterior edges of the implant sit between 1-3 mm from the anterior and posterior margins of the vertebral body endplate. The first patient-specific profile can also include a topography contoured to match (e.g., mate with) the topography of the first vertebral endplate determined at block 1604.

The operations at blocks 1608, 1610, and 1612 can generally correspond to the operations at blocks 1602, 1604, and 1606, except that they are directed to a second vertebral body endplate and a second patient-specific profile. The second vertebral body endplate can be opposite a disc space of the first vertebral body endplate. For example, if the first vertebral body endplate is the inferior endplate of the C2 vertebral body, the second vertebral body endplate can be the superior endplate of the C3 vertebral body. In embodiments in which the implant is a corpectomy device, the second vertebral body endplate can be the superior endplate of the C4 vertebral body endplate. The second patient-specific surface profile can be based on the determined lengths and topography of the second vertebral body endplate. Of note, as described throughout this Detailed Description, the second patient-specific surface profile is generally different in both size and topography than the first patient-specific surface profile.

After the first and second patient-specific surface profiles are generated, the method 1600 can continue at block 1614 by designing a patient-specific implant having the first patient-specific surface profile and the second patient-specific profile. For example, the implant can have a first (e.g., superior) endplate having the first patient-specific profile, and a second (e.g., inferior) endplate having the second patient-specific profile. The implant can then be manufactured and implanted into the patient in accordance with a patient-specific surgical plan.

### E. Conclusion

As one skilled in the art will appreciate, any of the software modules described previously may be combined into a single software module for performing the operations described herein. Likewise, the software modules can be distributed across any combination of the computing systems and devices described herein, and are not limited to the express arrangements described herein. Accordingly, any of the operations described herein can be performed by any of the computing devices or systems described herein, unless expressly noted otherwise.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, features, systems, devices, materials, methods and techniques described herein may, in some embodiments, be similar to any one or more of the embodiments, features, systems, devices, materials, methods and techniques described in the following:
U.S. application Ser. No. 16/048,167, filed on Jul. 27, 2018, titled “SYSTEMS AND METHODS FOR ASSISTING AND AUGMENTING SURGICAL PROCEDURES;”
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY;”
U.S. application Ser. No. 16/207,116, filed on Dec. 1, 2018, titled “SYSTEMS AND METHODS FOR MULTI-PLANAR ORTHOPEDIC ALIGNMENT;”
U.S. application Ser. No. 16/352,699, filed on Mar. 13, 2019, titled “SYSTEMS AND METHODS FOR ORTHOPEDIC IMPLANT FIXATION;”
U.S. application Ser. No. 16/383,215, filed on Apr. 12, 2019, titled “SYSTEMS AND METHODS FOR ORTHOPEDIC IMPLANT FIXATION;”
U.S. application Ser. No. 16/569,494, filed on Sep. 12, 2019, titled “SYSTEMS AND METHODS FOR ORTHOPEDIC IMPLANTS;” and
U.S. application Ser. No. 16/699,447, filed Nov. 29, 2019, titled “SYSTEMS AND METHODS FOR ORTHOPEDIC IMPLANTS;”
U.S. application Ser. No. 16/735,222, filed Jan. 6, 2020, titled “PATIENT-SPECIFIC MEDICAL PROCEDURES AND DEVICES, AND ASSOCIATED SYSTEMS AND METHODS;”
U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS;”
U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS;”
U.S. application Ser. No. 17/085,564, filed Oct. 30, 2020, titled “SYSTEMS AND METHODS FOR DESIGNING ORTHOPEDIC IMPLANTS BASED ON TISSUE CHARACTERISTICS;”
U.S. application Ser. No. 17/100,396, filed Nov. 20, 2020, titled “PATIENT-SPECIFIC VERTEBRAL IMPLANTS WITH POSITIONING FEATURES;”
U.S. application Ser. No. 17/342,439, filed Jun. 8, 2021, titled “PATIENT-SPECIFIC MEDICAL PROCEDURES AND DEVICES, AND ASSOCIATED SYSTEMS AND METHODS;”
U.S. application Ser. No. 17/463,054, filed Aug. 31, 2021, titled “BLOCKCHAIN MANAGED MEDICAL IMPLANTS;”
U.S. application Ser. No. 17/518,524, filed Nov. 3, 2021, titled “PATIENT-SPECIFIC ARTHROPLASTY DEVICES AND ASSOCIATED SYSTEMS AND METHODS;”
U.S. application Ser. No. 17/531,417, filed Nov. 19, 2021, titled “PATIENT-SPECIFIC JIG FOR PERSONALIZED SURGERY;”
U.S. application Ser. No. 17/678,874, filed Feb. 23, 2022, titled “NON-FUNGIBLE TOKEN SYSTEMS AND METHODS FOR STORING AND ACCESSING HEALTHCARE DATA;”
U.S. application Ser. No. 17/835,777, filed Jun. 8, 2022, titled “PATIENT-SPECIFIC EXPANDABLE INTERVERTEBRAL IMPLANTS;”
U.S. application Ser. No. 17/842,242, filed Jun. 16, 2022, titled “PATIENT-SPECIFIC ANTERIOR PLATE IMPLANTS;”
U.S. application Ser. No. 17/851,487, filed Jun. 28, 2022, titled “PATIENT-SPECIFIC ADJUSTMENT OF SPINAL IMPLANTS, AND ASSOCIATED SYSTEMS AND METHODS;”
U.S. application Ser. No. 17/856,625, filed Jul. 1, 2022, titled “SPINAL IMPLANTS FOR MESH NETWORKS;”
U.S. application Ser. No. 17/867,621, filed Jul. 18, 2022, titled “PATIENT-SPECIFIC SACROILIAC IMPLANT, AND ASSOCIATED SYSTEMS AND METHODS;”
U.S. application Ser. No. 17/868,729, filed Jul. 19, 2022, titled “SYSTEMS FOR PREDICTING INTRAOPERATIVE PATIENT MOBILITY AND IDENTIFYING MOBILITY-RELATED SURGICAL STEPS;”
U.S. application Ser. No. 17/951,085, filed Sep. 22, 2022, titled “SYSTEMS FOR MANUFACTURING AND PRE-OPERATIVE INSPECTING OF PATIENT-SPECIFIC IMPLANTS;”
U.S. application Ser. No. 17/978,673, filed Nov. 1, 2022, titled “SPINAL IMPLANTS AND SURGICAL PROCEDURES WITH REDUCED SUBSIDENCE, AND ASSOCIATED SYSTEMS AND METHODS;”
U.S. application Ser. No. 17/978,746, filed Nov. 1, 2022, titled “PATIENT-SPECIFIC SPINAL INSTRUMENTS FOR IMPLANTING IMPLANTS AND DECOMPRESSION PROCEDURES;”
U.S. application Ser. No. 18/102,444, filed Jan. 27, 2023, titled “TECHNIQUES TO MAP THREE-DIMENSIONAL HUMAN ANATOMY DATA TO TWO-DIMENSIONAL HUMAN ANATOMY DATA;”
U.S. application Ser. No. 18/113,573, filed Feb. 24, 2023, titled “PATIENT-SPECIFIC IMPLANT DESIGN AND MANUFACTURING SYSTEM WITH A DIGITAL FILING CABINET;”
U.S. application Ser. No. 18/120,979, filed Mar. 13, 2023, titled “MULTI-STAGE PATIENT-SPECIFIC SURGICAL PLANS AND SYSTEMS AND METHODS FOR CREATING AND IMPLEMENTING THE SAME;”
U.S. application Ser. No. 18/455,881, filed Aug. 25, 2023, titled “SYSTEMS AND METHODS FOR GENERATING MULTIPLE PATIENT-SPECIFIC SURGICAL PLANS AND MANUFACTURING PATIENT-SPECIFIC IMPLANTS;”
U.S. application Ser. No. 18/384,762, filed Oct. 28, 2023, titled “SYSTEMS AND METHODS FOR SELECTING, REVIEWING, MODIFYING, AND/OR APPROVING SURGICAL PLANS;”
U.S. application Ser. No. 18/537,600, filed Dec. 12, 2023, titled “PATIENT-SPECIFIC IMPLANT DESIGN AND MANUFACTURING SYSTEM WITH A REGULATORY AND REIMBURSEMENT MANAGER;”
International Application No. PCT/US24/10202, filed Jan. 3, 2024, titled “PATIENT-SPECIFIC SPINAL FUSION DEVICES AND ASSOCIATED SYSTEMS AND METHODS;”
U.S. application Ser. No. 18/408,409, filed Jan. 9, 2024, titled “SYSTEM FOR EDGE CASE PATHOLOGY IDENTIFICATION AND IMPLANT MANUFACTURING;”
U.S. application Ser. No. 18/408,452, filed Jan. 9, 2024, titled “SYSTEM FOR MODELING PATIENT SPINAL CHANGES;”
U.S. application Ser. No. 18/415,577, filed Jan. 17, 2024, titled “PATIENT-SPECIFIC IMPLANT DESIGN AND MANUFACTURING SYSTEM WITH A SURGICAL IMPLANT POSITIONING MANAGER;”
U.S. application Ser. No. 18/892,151, filed Sep. 20, 2024, titled “ROTATABLE AND SURGICAL APPROACH-SPECIFIC INTERVERTEBRAL IMPLANTS FOR FUSION TECHNIQUES;”
U.S. application Ser. No. 18/905,055, filed Oct. 2, 2024, titled “PATIENT-SPECIFIC SURGICAL POSITIONING GUIDES AND METHODS OF MAKING AND USING THE SAME;”
U.S. application Ser. No. 19/249,682, filed Jun. 25, 2025, titled “PATIENT-SPECIFIC SPINAL FUSION DEVICES AND ASSOCIATED SYSTEMS AND METHODS”; and
U.S. application Ser. No. 19/015,447, filed Jan. 9, 2025, titled “POSTERIOR FIXATION SYSTEMS FOR SPINAL TREATMENTS”.

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

The ranges disclosed herein also encompass any and all overlap, sub-ranges, and combinations thereof. Language such as “up to,” “at least,” “greater than,” “less than,” “between,” or the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

What is claimed is:

1. A patient-specific interbody implant for a patient, the implant comprising:
a body;
a first endplate configured to contact an inferior surface of a first vertebral body when the implant is implanted in the patient, wherein—
the first endplate includes a patient-specific topography designed to fit a corresponding topography of the inferior surface of the first vertebral body, and
the first endplate has a first size designed to cover at least 75% of a first surface area of the inferior surface of the first vertebral body; and
a second endplate configured to contact a superior surface of a second vertebral body when the implant is implanted in the patient, wherein—
the second endplate includes a patient-specific topography designed to fit a corresponding topography of the inferior surface of the second vertebral body, and
the second endplate has a second size designed to cover at least 75% of a second surface area the superior surface of the second vertebral body,
wherein the first size and the second size are different.

2. The implant of claim 1 wherein the second size is greater than the first size.

3. The implant of claim 1 wherein the first endplate has a first anteroposterior diameter and the second endplate has a second anteroposterior diameter, and wherein the first anteroposterior diameter is less than the second anteroposterior diameter.

4. The implant of claim 1 wherein the first endplate has a first transverse diameter and the second endplate has a second transverse diameter, and wherein the first transverse diameter is less than the second transverse diameter.

5. The implant of claim 1 wherein a cross-sectional area of the implant increases in the superior to inferior direction.

6. The implant of claim 5 wherein the cross-sectional area increases in a linear progression from the superior to inferior direction.

7. The implant of claim 5 wherein the cross-sectional area increases in an irregular progression from the superior to inferior direction.

8. The implant of claim 1 wherein the first size is greater than the second size.

9. The implant of claim 1 wherein the first size is designed to cover at least 90% of the first surface area, and wherein the second size is designed to cover at least 90% of the second surface area.

10. The implant of claim 1 wherein the first size is designed to cover about the same percentage of the first surface area as the second size is designed to cover the percentage of the second surface area.

11. The implant of claim 1 wherein the first size is designed to cover a different percentage of the first surface area by at least 5% as compared to the percentage of the second surface area that the second size is designed to cover.

12. The implant of claim 1 wherein the first endplate has a first shape, and wherein the second endplate has a second shape that is different than the first shape.

13. The implant of claim 1 wherein the body, the first endplate, and the second endplate form a unitary structure.

14. The implant of claim 1 wherein the body, the first endplate, and the second endplate are discrete components configured to be coupled together before being implanted in the patient.

15. The implant of claim 1 wherein the body is expandable.

16. The implant of claim 1 wherein the first vertebral body and the second vertebral body are immediately adjacent.

17. The implant of claim 1 wherein the first vertebral body and the second vertebral body are spaced apart by at least one intermediate vertebral body to be removed such that the implant is a corpectomy device.

18. The implant of claim 1 wherein the implant is a cervical implant designed to be implanted in the patient's cervical spine.

19. A computer-implemented method of designing a patient-specific interbody implant for a patient, the method comprising:
receiving image date of at least a portion of the patient's spine;
generating a digital representation of the patient's spine based at least in part on the image data;
adjusting the digital representation of the patient's spine to provide one or more corrections to the patient's spine, and
designing a patient-specific interbody implant to provide the one or more corrections to the patient's spine, wherein the patient-specific interbody implant includes —
a first endplate configured to contact an inferior surface of a first vertebral body when the implant is implanted in the patient, wherein the first endplate has a first size designed to cover at least 75% of a first surface area of the inferior surface of the first vertebral body, and
a second endplate configured to contact a superior surface of a second vertebral body when the implant is implanted in the patient, wherein the second endplate has a second size designed to cover at least 75% of a second surface area the superior surface of the second vertebral body,
wherein the first size and the second size are different.

20. The computer-implemented method of claim 19 wherein designing the patient-specific interbody implant includes:
generating a first profile of the inferior surface of the first vertebral body based on the digital representation of the patient's spine;
generating a second profile of the superior surface of the second vertebral body based on the digital representation of the patient's spine, wherein a size and a shape of the second profile is different than a size and a shape of the first profile;
determining the first size of the first endplate based on the first profile; and
determining the second size of the second endplate based on the second profile.

21. The computer-implemented method of claim 20 wherein:
generating the first profile includes determining a first anterior-posterior dimension and a first transverse dimension of the inferior surface from the digital representation; and
generating the second profile includes determining a second anterior-posterior dimension and second first transverse dimension of the inferior surface from the digital representation,
wherein the first anterior-posterior dimension is different than the second anterior-posterior dimension, and the first transverse dimensions is different than the second transverse dimension.

22. The computer-implemented method of claim 19 wherein the digital representation includes a three-dimensional virtual model.

23. The computer-implemented method of claim 19 wherein the second size is greater than the first size.

24. The computer-implemented method of claim 19 wherein the first endplate has a first anteroposterior diameter and the second endplate has a second anteroposterior diameter, and wherein the first anteroposterior diameter is less than the second anteroposterior diameter.

25. The computer-implemented method of claim 19 wherein the first endplate has a first transverse diameter and the second endplate has a second transverse diameter, and wherein the first transverse diameter is less than the second transverse diameter.

26. The computer-implemented method of claim 19 wherein a cross-sectional area of the implant increases in the superior to inferior direction.

27. The computer-implemented method of claim 19 wherein the first size is greater than the second size.

28. The computer-implemented method of claim 19 wherein the first size is designed to cover at least 90% of the first surface area, and wherein the second size is designed to cover at least 90% of the second surface area.

29. The computer-implemented method of claim 19 wherein the first size is designed to cover about the same percentage of the first surface area as the second size is designed to cover the percentage of the second surface area.

30. The computer-implemented method of claim 19 wherein the first size is designed to cover a different percentage of the first surface area by at least 5% as compared to the percentage of the second surface area that the second size is designed to cover.

31. The computer-implemented method of claim 19 wherein the first endplate has a first shape, and wherein the second endplate has a second shape that is different than the first shape.

32. The computer-implemented method of claim 19 wherein the first vertebral body and the second vertebral body are immediately adjacent.

33. The computer-implemented method of claim 19 wherein the first vertebral body and the second vertebral body are spaced apart by at least one intermediate vertebral body to be removed such that the implant is a corpectomy device.

34. The computer-implemented method of claim 19 wherein the implant is a cervical implant designed to be implanted in the patient's cervical spine.

35. A system for designing a patient-specific interbody implant for a patient, the system comprising:
one or more processors; and
one or more memories storing instructions that, when executed by the one or more processors, cause the system to perform a process for designing a patient-specific interbody implant for the patient, the process comprising:
receiving image date of at least a portion of the patient's spine;
generating a digital representation of the patient's spine based at least in part on the image data;
adjusting the digital representation of the patient's spine to provide one or more corrections to the patient's spine, and
designing a patient-specific interbody implant to provide the one or more corrections to the patient's spine, wherein the patient-specific interbody implant includes—
a first endplate configured to contact an inferior surface of a first vertebral body when the implant is implanted in the patient, wherein the first endplate has a first size designed to cover at least 75% of a first surface area of the inferior surface of the first vertebral body, and
a second endplate configured to contact a superior surface of a second vertebral body when the implant is implanted in the patient, wherein the second endplate has a second size designed to cover at least 75% of a second surface area the superior surface of the second vertebral body,
wherein the first size and the second size are different.

36. The system of claim 35 wherein the operation of designing the patient-specific interbody implant includes:
generating a first profile of the inferior surface of the first vertebral body based on the digital representation of the patient's spine;
generating a second profile of the superior surface of the second vertebral body based on the digital representation of the patient's spine, wherein a size and a shape of the second profile is different than a size and a shape of the first profile;
determining the first size of the first endplate based on the first profile; and
determining the second size of the second endplate based on the second profile.

37. The system of claim 36 wherein:
generating the first profile includes determining a first anterior-posterior dimension and a first transverse dimension of the inferior surface from the digital representation; and
generating the second profile includes determining a second anterior-posterior dimension and second first transverse dimension of the inferior surface from the digital representation,
wherein the first anterior-posterior dimension is different than the second anterior-posterior dimension, and the first transverse dimensions is different than the second transverse dimension.

38. The system of claim 35 wherein the digital representation includes a three-dimensional virtual model.

39. The system of claim 35 wherein the second size is greater than the first size.

40. The system of claim 35 wherein the first endplate has a first anteroposterior diameter and the second endplate has a second anteroposterior diameter, and wherein the first anteroposterior diameter is less than the second anteroposterior diameter.

41. The system of claim 35 wherein the first endplate has a first transverse diameter and the second endplate has a second transverse diameter, and wherein the first transverse diameter is less than the second transverse diameter.

42. The system of claim 35 wherein a cross-sectional area of the implant increases in the superior to inferior direction.

43. The system of claim 35 wherein the first size is greater than the second size.

44. The system of claim 35 wherein the first size is designed to cover at least 90% of the first surface area, and wherein the second size is designed to cover at least 90% of the second surface area.

45. The system of claim 35 wherein the first size is designed to cover about the same percentage of the first surface area as the second size is designed to cover the percentage of the second surface area.

46. The system of claim 35 wherein the first size is designed to cover a different percentage of the first surface area by at least 5% as compared to the percentage of the second surface area that the second size is designed to cover.

47. The system of claim 35 wherein the first endplate has a first shape, and wherein the second endplate has a second shape that is different than the first shape.

48. The system of claim 35 wherein the first vertebral body and the second vertebral body are immediately adjacent.

49. The system of claim 35 wherein the first vertebral body and the second vertebral body are spaced apart by at least one intermediate vertebral body to be removed such that the implant is a corpectomy device.

50. The system of claim 35 wherein the implant is a cervical implant designed to be implanted in the patient's cervical spine.

51. A patient-specific interbody implant for a patient, the implant comprising:
a body;
a first endplate configured to contact an inferior surface of a first vertebral body when the implant is implanted in the patient, wherein—
the first endplate includes a patient-specific topography designed to fit a corresponding topography of the inferior surface of the first vertebral body, and
the first endplate has a first size and a first shape designed to at least partially cover a surface area of the inferior surface of the first vertebral body; and
a second endplate configured to contact a superior surface of a second vertebral body when the implant is implanted in the patient, wherein—
the second endplate includes a patient-specific topography designed to fit a corresponding topography of the inferior surface of the second vertebral body, and
the second endplate has a second size and a second shape designed to at least partially cover a surface area the superior surface of the second vertebral body,
wherein the first size and the second size are different and the first shape and the second shape are different.

52. The implant of claim 51 wherein a cross-sectional area of the implant increases in the superior to inferior direction.

53. The implant of claim 51 wherein the first endplate is designed to cover at least about 80% of the surface area of the inferior surface of the first vertebral body, and wherein the second endplate is designed to cover at least about 80% of the surface area of the superior surface of the second vertebral body.

54. The implant of claim 51 wherein the first shape is a circular, rectangular, and/or oblong shape.

55. A computer-implemented method of designing a patient-specific interbody implant for a patient, the method comprising:
receiving image date of the patient's spine;
generating a digital representation of at least a portion of the patient's spine based at least in part on the image data, wherein the portion of the patient's spine includes—
a first vertebral body with a first central region and a first cortical rim surrounding the first central region, and
a second vertebral body with a second central region and a second cortical rim surrounding the second central region; and
designing a patient-specific interbody implant based on the digital representation, wherein the patient-specific interbody implant includes—
a first endplate configured to contact a first threshold load-bearing amount of the first central region and to be surrounded by the first cortical rim when implanted, and
a second endplate configured to contact a second threshold load-bearing amount of the second central region and to be surrounded by the second cortical rim when implanted,
wherein the first endplate has a first size and the second endplate has a second size different than the first size.

56. The method of claim 55, further comprising:
determining a loading capability of the first vertebral body and the second vertebral body; and
determining the threshold load-bearing amount of the first central region and the second central region based on the loading capability of the first vertebral body and the second vertebral body.

57. The method of claim 55, further comprising receiving user input indicating the first threshold load-bearing amount and the second threshold load-bearing amount.

58. The method of claim 55 wherein the first threshold load-bearing amount and the second threshold load-bearing amount are different.

59. The method of claim 55 wherein the first threshold load-bearing amount is at least 75% of a first surface area of the first central region, and wherein the second threshold load-bearing amount is at least 75% of a second surface area of the second central region.

60. The method of claim 55, further comprising:
simulating loading of the portion of the patient's spine when the patient-specific implanted is implanted; and
sending output from the simulation for viewing by a user to evaluate the patient-specific interbody implant.

61. The method of claim 55, further comprising:
simulating subsidence of the patient-specific implant; and
at least partially redesigning the patient-specific interbody implant based on the simulation.

62. The method of claim 55, further comprising:
simulating bone ingrowth in the patient-specific implant; and
at least partially redesigning the patient-specific interbody implant based on the simulation.

63. The method of claim 55, further comprising:
retrieving a target outcome for the patient using a machine learning model; and
performing one or more stress analyses of the first and/or second vertebral body to determine the threshold load-bearing amount that achieves the target outcome.

64. A system for designing a patient-specific interbody implant for a patient, the system comprising:
one or more processors; and
one or more memories storing instructions that, when executed by the one or more processors, cause the system to perform a process for designing a patient-specific interbody implant for the patient, the process comprising:
receiving image date of the patient's spine;
generating a digital representation of at least a portion of the patient's spine based at least in part on the image data, wherein the portion of the patient's spine includes—
a first vertebral body with a first central region and a first cortical rim surrounding the first central region, and
a second vertebral body with a second central region and a second cortical rim surrounding the second central region; and
designing a patient-specific interbody implant based on the digital representation, wherein the patient-specific interbody implant includes —
a first endplate configured to contact a first threshold load-bearing amount of the first central region and to be surrounded by the first cortical rim when implanted, and
a second endplate configured to contact a second threshold load-bearing amount of the second central region and to be surrounded by the second cortical rim when implanted,
wherein the first endplate has a first size and the second endplate has a second size different than the first size.

65. The system of claim 64 wherein the process further comprises:
determining a loading capability of the first vertebral body and the second vertebral body; and
determining the threshold load-bearing amount of the first central region and the second central region based on the loading capability of the first vertebral body and the second vertebral body.

66. The system of claim 64 wherein the process further comprises receiving user input indicating the first threshold load-bearing amount and the second threshold load-bearing amount.

67. The system of claim 64 wherein the first threshold load-bearing amount and the second threshold load-bearing amount are different.

68. The system of claim 64 wherein the first threshold load-bearing amount is at least 75% of a first surface area of the first central region, and wherein the second threshold load-bearing amount is at least 75% of a second surface area of the second central region.

69. The system of claim 64 wherein the process further comprises:
simulating loading of the portion of the patient's spine when the patient-specific implanted is implanted; and
sending output from the simulation for viewing by a user to evaluate the patient-specific interbody implant.

70. The system of claim 64 wherein the process further comprises:
simulating subsidence of the patient-specific implant; and
at least partially redesigning the patient-specific interbody implant based on the simulation.

71. The system of claim 64 wherein the process further comprises:
simulating bone ingrowth in the patient-specific implant; and
at least partially redesigning the patient-specific interbody implant based on the simulation.

72. The system of claim 64 wherein the process further comprises:
retrieving a target outcome for the patient using a machine learning model; and
performing one or more stress analyses of the first and/or second vertebral body to determine the threshold load-bearing amount that achieves the target outcome.

73. A surgical kit, comprising:
a first interbody implant having a first endplate and a second endplate;
a second interbody implant having a third endplate and a fourth endplate; and
a third interbody implant having a fifth endplate and a sixth endplate,
wherein each of the first, second, third, fourth, fifth, and sixth endplates have different sizes such that no endplate of any implant in the kit is the same size as any other endplate in the kit.

74. The surgical kit of claim 73 wherein each of the first, second, third, fourth, fifth, and sixth endplates have a patient topography.

75. The surgical kit of claim 73 wherein each of the first, second, third, fourth, fifth, and sixth endplates are designed to cover at least 75% of a corresponding vertebral body endplate surface.

76. The surgical kit of claim 73 wherein:
the first implant is designed to be implanted within a first intervertebral segment of a spine of a patient,
the second implant is designed to be implanted within a second intervertebral segment of the spine of the patient, and
the third implant is designed to be implanted within a third intervertebral segment of the spine of the patient,
wherein each of the first intervertebral segment, the second intervertebral segment, and third intervertebral segment are different.

77. The surgical kit of claim 76 wherein the first intervertebral segment, the second intervertebral segment, and the third intervertebral segment are between C1 and C6.

78. The surgical kit of claim 76, further comprising an anterior plate configured for use with the first implant, the second implant, and the third implant, wherein the anterior plate is configured to extend across the first intervertebral segment, the second intervertebral segment, and the third intervertebral segment.

79. The surgical kit of claim 78 wherein the anterior plate has a concave posterior surface having a patient-specific curvature.

80. A patient-specific interbody implant for a patient, the implant comprising:
a body;
a first endplate configured to contact an inferior surface of a first vertebral body when the implant is implanted in the patient, wherein the first endplate includes—
a first patient-specific topography designed to fit a corresponding topography of the inferior surface of the first vertebral body, and
a first cortical rim receiving portion having a first load-bearing seating portion and a first semi-curved perimeter ridge extending in a superior direction from the first load-bearing seating portion; and
a second endplate configured to contact a superior surface of a second vertebral body when the implant is implanted in the patient, wherein the second endplate includes—
a second patient-specific topography designed to fit a corresponding topography of the superior surface of the second vertebral body, and
a second cortical rim receiving portion having a second load-bearing seating portion and a second semi-curved perimeter ridge extending in an inferior direction from the second load-bearing seating portion.

81. The patient-specific interbody implant of claim 80 wherein:
the first semi-curved perimeter ridge extends between about 1 mm and about 5 mm superiorly relative to the first load-bearing seating portion, and
the second semi-curved perimeter ridge extends between about 1 mm and about 5 mm inferiorly relative to the second load-bearing seating portion.

82. The patient-specific interbody implant of claim 80 wherein the first semi-curved perimeter ridge and the second semi-curved perimeter ridge extend by different magnitudes relative to the first load-bearing seating portion and the second load-bearing seating portion, respectively.

83. The patient-specific interbody implant of claim 80 wherein:
the first semi-curved perimeter ridge extends around at least 75% of a perimeter of the first endplate, and
the second semi-curved perimeter ridge extends around at least 75% of a perimeter of the second endplate.

84. The patient-specific interbody implant of claim 80 wherein the first load-bearing seating portion is configured to contact a cortical rim of the superior vertebral body, and wherein the second load-bearing seating portion is configured to contact a cortical rim of the inferior vertebral body.

85. A patient-specific interbody implant for a patient, the implant comprising:
a first endplate configured to contact an inferior surface of a first vertebral body when the implant is implanted in the patient, wherein the first endplate includes a first patient-specific topography designed to fit a corresponding topography of the inferior surface of the first vertebral body;
a second endplate configured to contact a superior surface of a second vertebral body when the implant is implanted in the patient, wherein the second endplate includes a second patient-specific topography designed to fit a corresponding topography of the superior surface of the second vertebral body;
an anterior surface extending between the first endplate and the second endplate;
a posterior surface extending between the first endplate and the second endplate; and
a wing-like extension at an anterior portion of the implant and extending inferiorly relative to a plane of the second endplate such that a first height of the anterior surface is between about 50% and about 200% greater than a second height of posterior surface.

86. The patient-specific interbody implant of claim 85 wherein the wing-like extension has a third height that is equal to or greater than the second height.

87. The patient-specific interbody implant of claim 85 wherein the wing-like extension conforms to a shape of a portion of the second vertebral body.

88. The patient-specific interbody implant of claim 85 wherein the first endplate has a first size designed to cover at least 75% of a first surface area of the inferior surface of the first vertebral body, and the second endplate has a second size designed to cover at least 75% of a second surface area of the superior surface of the second vertebral body, wherein the first size and the second size are different.

89. A patient-specific implant system, the system comprising:
an interbody implant sized and shaped to be implanted within an intervertebral disc space of a patient, wherein the interbody implant includes—
a superior surface having a first patient-specific topography,
an inferior surface having a second patient-specific topography, and
an anterior surface extending between the superior surface and the inferior surface, wherein the anterior surface includes a cavity, the cavity including an aperture configured to receive an inserter instrument, a first recessed portion extending in a first direction relative to the aperture, and a second recessed portion extending in a second direction relative to the aperture; and
a plate sized and shaped to be implanted at an anterior portion of a vertebral column of the patient, wherein the plate includes—
an anterior surface,
a posterior surface,
one or more openings extending between the anterior surface and the posterior surface and sized and shaped to receive one or more fixation elements for coupling the plate to the vertebral column, and
a plurality of coupling features extending from the posterior surface of the plate, the plurality of coupling features including a first coupling feature sized and shaped to sit within the first recessed portion of the interbody implant and a second coupling feature sized and shaped to sit within the second recessed portion of the interbody implant,
wherein the interbody implant and the plate are not fixedly coupled together when the coupling features are inserted into the first and second recessed portions to permit micromovements therebetween.

90. The patient-specific implant system of claim 89 wherein the anterior surface does not include any holes configured to receive a fixation element.

91. The patient-specific implant system of claim 89 wherein the plate further includes a retention mechanism configured to retain the one or more fixation elements within the one or more openings.

92. The patient-specific implant system of claim 91, wherein the retention mechanism incudes a cam rotatable between a first, unlocked configuration in which the cam does not block the one or more openings, and a second, locked configuration in which the cam at least partially blocks the one or more openings.

93. The patient-specific implant system of claim 92 wherein the anterior surface of the plate includes a recess, and wherein the cam rotates within the recess such that the cam does not extend anteriorly beyond an anterior surface of the plate.

94. The patient-specific implant system of claim 91, wherein the retention mechanism includes a central opening extending therethrough and configured to receive an inserter instrument.

95. The patient-specific implant system of claim 94 wherein, when the coupling features are inserted into the first and second recessed portions, the central opening of the plate and the aperture in the anterior surface of the interbody implant share a common central axis.

96. The patient-specific implant system of claim 89 wherein:
the first coupling feature has a different size and/or shape than the second coupling feature, and
the first recessed portion has a different size and/or shape than the second recessed portion.

97. The patient-specific implant system of claim 96 wherein the first coupling feature is complementary to the first recessed portion, and wherein the second coupling feature is complementary to the second recessed portion.
